(19) United States
(12) Patent Application Publication
(10) Pub. No.: US 2013/0218024 A1
(43) Pub. Date: Aug. 22, 2013

(54) Interventional In-Situ Image-Guidance by Fusing Ultrasound and Video

(71) Applicant: CLEAR GUIDE MEDICAL, LLC, (US)

(72) Inventors: Emad Mikhail BOCTOR, Baltimore, MD (US); Gregory Donald HAGER, Baltimore, MD (US); Philipp Jakob STOLKA, Baltimore, MD (US); Dorothee HEISENBERG, Baltimore, MD (US)

(73) Assignee: CLEAR GUIDE MEDICAL, LLC, Baltimore, MD (US)

(21) Appl. No.: 13/648,245

(22) Filed: Oct. 9, 2012

Related U.S. Application Data
(60) Provisional application No. 61/545,186, filed on Oct. 9, 2011.
(60) Provisional application No. 61/603,625, filed on Feb. 27, 2012.
(60) Provisional application No. 61/657,441, filed on Jun. 8, 2012.

(51) Int. Cl. A61B 19/00; A61B 6/00; A61B 8/08; A61B 8/00; A61B 19/08; A61B 5/06; A61B 5/055; A61B 5/00; A61B 6/03

(52) U.S. Cl. CPC A61B 19/5244; A61B 5/0077; A61B 5/742; A61B 6/4441; A61B 8/488; A61B 6/032; A61B 19/088; A61B 8/0841; A61B 5/062; A61B 5/055; A61B 8/4416; A61B 5/0075
USPC 600/476

(57) ABSTRACT

An augmentation device for an imaging system has a bracket structured to be attachable to an imaging component, and a projector attached to the bracket. The projector is arranged and configured to project an image onto a surface in conjunction with imaging by the imaging system. A system for image-guided surgery has an imaging system, and a projector configured to project an image or pattern onto a region of interest during imaging by the imaging system. A capsule imaging device has an imaging system, and a local sensor system. The local sensor system provides information to reconstruct positions of the capsule endoscope free from external monitoring equipment.

### CROSS-REFERENCE OF RELATED APPLICATION

This application claims priority to U.S. Provisional Application No. US61/545,168 filed Oct. 9, 2011, U.S. Provisional Application No. US61/603,625, filed Feb. 27, 2012, and U.S. Provisional Application No. US61/657,441, filed Jun. 8, 2012, the entire contents of which are hereby incorporated by reference.

### BACKGROUND

1. Field of Invention

The field of the currently claimed embodiments of this invention relate to imaging devices and to augmentation devices for these imaging devices, and more particularly to such devices that have one or more of a camera, one or more of a projector, and/or a set of local sensors for observation and imaging of, projecting onto, and tracking within and around a region of interest.

2. Discussion of Related Art

Image-guided surgery (IGS) can be defined as a surgical or intervention procedure where the doctor uses indirect visualization to operate, i.e. by employing imaging instruments in real time, such as fiber-optic guides, internal video cameras, flexible or rigid endoscopes, ultrasonography etc. Most image-guided surgical procedures are minimally invasive. IGS systems allow the surgeon to have more information available at the surgical site while performing a procedure. In general, these systems display 3D patient information and render the surgical instrument in this display with respect to the anatomy and a preoperative plan. The 3D patient information can be a preoperative scan such as CT or MRI to which the patient is registered during the procedure, or it can be a real-time imaging modality such as ultrasound or fluoroscopy. Such guidance assistance is particularly crucial for minimally invasive surgery (MIS), where a procedure or intervention is performed either through small openings in the body or percutaneously (e.g. in ablation or biopsy procedures). MIS techniques provide for reductions in patient discomfort, healing time, risk of complications, and help improve overall patient outcomes.

In image-guided interventions, the tracking and localization of imaging devices and medical tools during procedures are exceptionally important and are considered the main enabling technology in IGS systems. Tracking technologies can be easily categorized into the following groups: 1) mechanical-based tracking including active robots (DaVinci robots [http://www.intuitivesurgical.com, Aug. 2, 2010]) and passive-encoded mechanical arms (Faro mechanical arms [http://products.faro.com/product-overview, Aug. 2, 2010]), 2) optical-based tracking (NDI OptoTrak [http://www.ndigital.com, Aug. 2, 2010], MicronTracker [http://www.clarontech.com, Aug. 2, 2010]), 3) acoustic-based tracking, and 4) electromagnetic (EM)-based tracking (Ascension Technology [http://www.ascension-tech.com, Aug. 2, 2010]).

Ultrasound is one useful imaging modality for image-guided interventions including ablative procedures, biopsy, radiation therapy, and surgery. In the literature and in research labs, ultrasound-guided intervention research is performed by integrating a tracking system (either optical or EM methods) with an ultrasound (US) imaging system to, for example, track and guide liver ablations, or in external beam radiation therapy [E. M. Boctor, M. DeOliviera, M. Choti, R. Ghanem, R. H. Taylor, G. Hager, G. Fichtinger, “Ultrasound Monitoring of Tissue Ablation via Deformation Model and Shape Priors”, International Conference on Medical Image Computing and Computer-Assisted Intervention, MICCAI 2006; H. Rivaz, I. Fleming, L. Assumpcao, G. Fichtinger, U. Hamper, M. Choti, G. Hager, and E. Boctor, “Ablation monitoring with elastography: 2D in-vivo and 3D ex-vivo studies”, International Conference on Medical Image Computing and Computer-Assisted Intervention, MICCAI 2008; H. Rivaz, P. Foroughi, I. Fleming, R. Zellars, E. Boctor, and G. Hager, “Tracked Regularized Ultrasound Elastography for Targeting Breast Radiotherapy”, Medical Image Computing and Computer Assisted Intervention (MICCAI) 2009]. On the commercial side, Siemens and GE Ultrasound Medical Systems recently launched a new interventional system, where an EM tracking device is integrated into high-end cart-based systems. Small EM sensors are integrated into the ultrasound probe, and similar sensors are attached and fixed to the intervention tool of interest.

Limitations of the current approach on both the research and commercial sides can be attributed to the available tracking technologies and to the feasibility of integrating these systems and using them in clinical environments. For example, mechanical-based trackers are considered expensive and intrusive solutions, i.e. they require large space and limit user motion. Acoustic tracking does not provide sufficient navigation accuracy, leaving optical and EM tracking as the most successful and commercially available tracking technologies. However, both technologies require intrusive setups with a base camera (in case of optical tracking methods) or a reference EM transmitter (in case of EM methods). Additionally, optical rigid-body or EM sensors have to be attached to the imager and all needed tools, hence require offline calibration and sterilization steps. Furthermore, none of these systems natively assist multi-modality fusion (registration e.g. between pre-operative CT/MRI plans and intra-operative ultrasound), and do not contribute to direct or augmented visualization either. Thus there remains a need for improved imaging devices for use in image-guided surgery.

### SUMMARY OF THE INVENTION

An augmentation device for an imaging system according to an embodiment of the current invention has a bracket structured to be attachable to an imaging component, a projector attached to the bracket, and one or more cameras observing the surrounding environment. The projector is arranged and configured to project an image onto a surface in conjunction with imaging by the camera system. This system can be used for registration to the imaged surface, and guidance for placement of the device on the surface, or guidance of needles or other instruments to interact with the surface or below the surface.

A system that consists of a single camera and project, whereby one of the camera or projector is aligned with the ultrasound plane, and the other is off-axis, and a combination of tracking and display is used to provide guidance.

The camera and projector configuration can be preserved using sterile probe covering that contain special transparent sterile window.

A structured pattern that simultaneously display the ultrasound image and also used to reconstruct the surface in 3D

The projection image may be time-multiplexed in synchrony with the camera or cameras to alternatively optimize projection for tracking (maximize needle presence), guidance (overlay clues), surfaces (optimize stereo reconstruction). The projection pattern may also be spatially modulated or multiplexed for different purposes, e.g. projecting a pattern in one area and guidance in other areas.

An adaptive pattern both in space and time including the following:

Spatial frequencies of the pattern to adopt surface distance, apparent structure sizes or camera resolution, or
Colors to adapt surface properties and environment, or
Randomize/iterate through different pattern overtime
Both pattern and projected guidance can be integrated and optimized to reconstruct surfaces

Real-time feedback and quality control system to choose actively the right pattern design.

Calculating system metrics—tracking success, robustness, surface outlier ratio to choose the right pattern.

A method to guide tool by actively tracking the tool and projecting:
proximity markers (to indicate general “closeness” by e.g. color-changing backgrounds, frames, or image tints, or auditory cues),
target markers (to point towards e.g. crosshairs, circles, bulls-eyes etc.),
alignment markers (to line up with e.g. lines, fans, polygons,
area demarcations (to avoid e.g. shapes denoting critical regions, geometrically or anatomically inaccessible regions etc.).
Patterns from circles on the edges of the field of view to allow other information to be projected inside the center of the field of view
Combination of the above

The guidance to be on screen or projected to the patient or combination of both; we claim the guidance method to be either separate or as an overlay to a secondary imaging system, such as ultrasound images or mono- or multi-ocular views.

This guidance approach and information to be either registered to the underlying image or environment (i.e. the overlay symbols correspond to target location, size, or areas to avoid); or it can be location-independent guidance (e.g. location, color, size, shape, but also auditory cues such as audio volume, sound clips, and/or frequency changes indicate to the user where to direct the tools or the probe.)

The combination of the camera and projector can be used to construct intuitive and sterile user interfaces on the patient surface, or on any other projectable surface. For example, standard icons and buttons can be projected onto the patient, and a finger or needle can be tracked and used to activate these buttons. This tracking can also be used in non-visual user interfaces, e.g. for gesture tracking without projected visual feedback.

The projection system may make use of the geometry computed by the stereo system to correct for the curvature of the body when projecting information onto it.

The system can include overlay guidance to place the imaging device on a surface (e.g. Ultrasound probe) or move it to a specific pose (e.g. C-arm X-ray). For example, by making use of the ability of an ultrasound probe or similar imaging device to acquire images from within the body while the video imaging system captures images from outside the body, it is possible to register the probe in body coordinates, and to project guidance as to how to move the probe to visualize a given target. For example, suppose that a tumor is identified in a diagnostic image, or in a previous scan. After registration the projection system can project an arrow on the patient showing in which direction the probe should move. One of ordinary skill will realize that these same ideas can be used to guide a user to visualize a particular organ based on a prior model of the patient or a patient-specific scan, or could be used to aid in tracking or orienting relative to a given target. For example, it may be desirable to place a gating window (e.g. for Doppler ultrasound) on a particular target or to maintain it therein.

It is often the case that a patient is imaged multiple times, for example to provide guidance for radiative cancer therapy. In this case, the images around the target could be recorded, and, upon subsequent imaging, these images would be used to provide guidance on how to move the probe toward a desired target, and an indication when the previous imaging position is reached.

A method to guide interventional tool by matching the tool's shadow to an artificial shadow—this single-shadow alignment can be used for one degree of freedom with additional active tracking for remaining degrees of freedom. The shadow can be a single line; the shadow can be a line of different thickness; the shadow can be of different colors; the shadow can be used as part of structured light pattern.

Adaptive projection to overcome interference (e.g. overlay guidance can interfere with needle tracking tasks): guidance “lines” composed of e.g. “string-of-pearls” series of circles/discs/ellipses etc. can improve alignment performance for the user.

Additionally, the apparent thickness of guidance lines/structures can be modified based on detected tool width, distance to projector, distance to surface, excessive intervention duration etc. to improve alignment performance

A method based on double shadow or more depending on the number of projectors or virtual projectors available

Two projectors can uniquely provide two independent shadows that can define the intended/optimal guide of the tool

Using a combination of mirrors and a beam splitter—one projector can be divided into two projectors and hence provide the same number of independent shadows

A method of guidance to avoid critical structure—by projecting onto patient surface information registered from pre-operative modality

A guidance system (one example)—Overlaying crosshairs and/or extrapolated needle pose lines onto live ultrasound views on-screen (both in-plane and out-of-plane) or projected onto the patient, see, e.g., FIG. 34; Projecting paired symbols (circles, triangles etc.) that change size, color, and relative position depending on the current targeting error vector; Overlaying alignment lines onto single/stereo/multiple camera views that denote desired needle poses, allowing the user to line up the camera image of the needle with the target pose, as well as lines denoting the currently-tracked needle pose for quality control purposes; Projecting needle alignment lines onto the surface, denoting both target pose (for guidance) as well as currently-tracked pose (for quality control), from one or more projectors;

The system may use the pose of the needle in air to optimize ultrasound to detect the needle in the body and vice-versa. For example, by expecting the location of the needle tip—the ultrasound system can automatically set the transmit focus location and the needle steering parameters etc.

When using the projector for needle guidance, the system may make use of the projected insertion point as “capture range” for possible needle poses, discard candidates outside that range, or detect when computed 3D poses violate the expected targeting behavior.

An approach to indicate depth of penetration of the tool. This can be performed by detecting fiducials on the needle, and tracking those fiducials over time. For example, these may be dark rings on the needle itself, which can be counted using the vision system, or they may be a reflective element attached to the end of the needle, and the depth may be computed by subtracting the location of the fiducial in space from the patient surface, and then subtracting that result from the entire length of the needle.

Depth guidance by directly projecting on the needle shaft a fiducial landmark (e.g. black line or spot of light), indicating to what point the needle should be inserted.

Additional depth guidance claim can be simply the display of the system may passively indicate the number of fiducial rings that should remain outside the patient at the correct depth for the current system pose, providing the user with a perceptual cue that they can use to determine manually if they are at the correct depth.

An apparatus and method to provide adaptable mounting bracket:
The camera/projector configuration can rotate 90 degrees to allow the guidance for both in-plane and out-of-plane intervention
The bracket height can be adjusted
The mounting bracket can be modular to add cameras, projectors, for example start with one projector and add one camera, or start with one projector and two cameras and add additional projector

The camera and projector can be added at different location (camera and projector for in-plane intervention and adding one projector facing the out-of-plane view)

A calibration method that simultaneously calibrates US, projector and stereo cameras. The method is based on a calibration object constructed from a known geometry:
Double-wedge phantom attached to a planar surface (as in FIG. 26A), or Multi-line phantom (as in FIG. 26B). Both are alternative designs of possible phantoms that can be used in estimating the rigid-body transformation between ultrasound coordinate frame and camera coordinates frame. In principle, a phantom with a well-known geometry comprising an ultrasound-visible component and an optically-visible component (as in FIGS. 26A and 26B) is simultaneously observed in both modalities. Pose recovery of both components in their respective modality allows reconstruction of the pose of the cameras and the ultrasound transducer relative to the phantom, and thus the calibration of their relative pose to the each other. See also, FIG. 33.
Multi-line with a known geometry connected to the surface and observed by cameras
Complex shape phantom with known geometry from a previous volumetric scan. By registering both live surface/US data with corresponding preoperative data, we can calibrate the system
Features in phantoms can be introduced by using nanocapsules that can rupture with ultrasound waves and create a visible mark (observed by cameras)

A method to accurately measure the location of the projector relative to the location of the cameras and probe. One means of doing so is to observe that visible rays projected from the camera will form straight lines in space that intersect at the optical center of the projector. Thus, with stereo cameras or a similar imaging system observing several surfaces upon which these rays fall, the system can calculate a series of 3D points which can then be extrapolated to compute the center of projection. This can be performed with nearly any planar or nonplanar series of projection surfaces.

A temporal calibration method that simultaneously synchronize ultrasound data stream to both cameras streams and to projector streams:

Calibration can be performed using hardware trigger approach

Software approach can be utilized by moving the US probe periodically above a target—correlating both streams should estimate the amount of internal lag

A method to synchronize projection output to allow time and space multiplexing (interleaving) patterns for both guidance and stereo structures.

A system that utilizes custom-made drapes with the following features:
Drapes that are transparent to the structured light system
Drapes that are IR transparent or wavelength-specific to allow patients surface or organ scanning
Drapes that made of texture or detectable reference frame to allow direct surface tracking and registration/reconstruction
Drapes that made of light sensitive materials utilizing Fluorescence and/or Phosphorescence effects. To help create an interface for the user to click on.
Drapes that are pressure sensitive—color changes with probe pressure or with changes in pressure due to breathing

The projector may make use of light-activated dyes that have been “printed on patient” or may contain an auxiliary controlled laser for this purpose.

A depth imaging system composed from more than two cameras. For example with three cameras where camera 1 and 2 are optimized for far range, camera 2 and 3 for mid-range, and camera 1 and 3 for close range.

An augmentation hardware to the original apparatus depending on the application. The overall configuration may be augmented by and/or controlled from a hand-held device such as a tablet computer for 1) ultrasound machine operation, 2) for visualization; 3) in addition, by using an one or more cameras on the tablet computer, for registration to patient for transparent information overlay.

An augmentation hardware to construct a display system that maintains registration with the probe and which can be used for both visualization and guidance. For example, the probe may have an associated display that the can be detached and which shows relevant pre-operative CT information based on its position in space. It may also overlay targeting information.

The computational resources used by the device may be augmented with additional computation located elsewhere.

This remote computation might be used to process information coming from the device (e.g. to perform a computationally intense registration process); it may be used to recall information useful to the function of the device (e.g. to compare this patient with other similar patients to provide “best practice” treatment options), or it may be used to provide information that directs the device (e.g. transferring the indication of a lesion in a CT image to a remote center for biopsy).

Quality control method for the overall system performance. The trajectory of a needle can be calculated by visual tracking and thence projected into the ultrasound image. If the needle in the image is inconsistent with this projection, it is a cue that there is a system discrepancy. Conversely, if the needle is detected in the ultrasound image, it can be projected back into the video image to confirm that the external pose of the needle is consistent with that tracked image.

Active quality control method by to simultaneously track the needle in both ultrasound and video images, and to use those computed values to detect needle bending and to either update the likely trajectory of the needle, or to alert the user that they are putting pressure on the needle, or both.

A guidance system based on camera/projector simultaneous interaction. In one embodiment, the projection center may lie on or near the plane of the ultrasound system. In this case, the projector can project a single line or shadow that indicates where this plane is. A needle or similar tool placed in the correct plane will become bright. A video camera outside this plane can view the scene, and this image can be displayed on a screen. Indeed, it may be included with the ultrasound view. In this case, the clinician can view both the external and internal guidance of the needle simultaneously on the same screen. Guidance to achieve a particular angle can be superimposed on the camera image, so that the intersection of the ultrasound plane and the plane formed by the superimposed guidance forms a line that is the desired trajectory of the needle.

A second embodiment of the simultaneous camera/projector guidance. A variation on this would be to place a camera along the ultrasound plane, and to place the projector off-plane. The geometry is similar, but now the camera superimposed image is used to define the plane, and a line is projected by the projector to define the needle trajectory.

Further variations include combinations of single or multiple cameras or projectors, where at least one of either is mounted on the mobile device itself as well as mounted statically in the environment, with registration between the mobile and fixed components maintained at all times to make guidance possible. This registration maintenance can be achieved e.g. by detecting and tracking known features present in the environment and/or projected into the common field of interest.

An augmentation system that may use multi-band projection with both visible and invisible bands (such as with IR in various ways), simultaneously or time-multiplexed. As noted above, the invention may use multi-projector setups for shadow reduction, intensity enhancement, or passive stereo guidance.

An augmentation device with stereo projection. In order to create a stereo projection, the projection system may make use of mirrors and splitters for making one projector two (or more) by using “arms” etc. to split the image or to accomplish omnidirectional projection.

The projection system may make use of polarization for 3D guidance or use dual-arm or dual-device projection with polarized light and (passive) glasses for 3D in-situ ultrasound guidance display. The projection may project onto a screen consisting of any of: Fog screen, switchable film, UV-fluorescent glass as almost-in-situ projection surfaces

An augmentation device where one of the cameras or a dedicated camera is outward-looking to track the user to help correct visualization from geometric distortion or probe motion. This may also be used to solve the parallax problem when projecting in 3D.

The augmentation device can estimate relative motion. The projection system may project a fixed pattern upwards onto the environment to support tracking with stereo cameras (limited degrees of freedom, depending on environment structure and the direction of motion)

A projection system that in addition of projecting on the patient surface; the projector might instead project onto other rigid or deformable objects in the workspace or the reading room. For example, the camera might reconstruct a sheet of paper in space, and the projector could project the CT data of a preoperative scan onto the paper. As the paper is deformed the CT data would be altered to reflect the data that it would “slice through” if it were inside the body. This would allow the visualization of curved surfaces or curvilinear structures.

A data entry approach that can improve the usability of guidance methods, the system may have an electronic or printable signature that records the essential targeting information in an easy-to-use way. This information may be loaded or scanned visually by the device itself when the patient is re-imaged.

An approach that benefit from conventional database and new visual database (enabled by the described technology) and provide unique training targeted to needed population.

This may include providing training for those learning about diagnostic or interventional ultrasound; or to make it possible for the general population to make use of ultrasound-based treatments for illness (automated carotid scanning in pharmacies).

These methods could also monitor the use of an imaging probe and/or needles etc. and indicate when the user is poorly trained.

There are many other applications for these ideas that extend beyond ultrasound and medicine. For example, nondestructive inspection of a plane wing may use ultrasound or x-ray, but in either case requires exact guidance to the inspection location (e.g. a wing attachment) in question. The methods described above can provide this guidance. In a more common setting, the system could provide guidance for e.g. throwing darts, hitting a pool ball, or a similar game.

### BRIEF DESCRIPTION OF THE DRAWINGS

Further objectives and advantages will become apparent from a consideration of the description, drawings, and examples.

FIG. 1 shows an embodiment of an augmentation device for an imaging system according to an embodiment of the current invention.

FIG. 2 is a schematic illustration of the augmentation device of FIG. 1 in which the bracket is not shown.

FIG. 3A is a schematic illustration of an augmentation device and imaging system according to an embodiment of the current invention.

FIG. 3B is a schematic illustration of an augmentation device and imaging system according to another embodiment of the invention.

FIG. 3C is a schematic illustration of an augmentation device and imaging system according to another embodiment of the invention.

FIG. 3D is a schematic illustration of an augmentation device and imaging system according to another embodiment of the invention.

FIG. 3E is a schematic illustration of an augmentation device and imaging system according to another embodiment of the invention.

FIG. 3F is a schematic illustration of an augmentation device and imaging system according to another embodiment of the invention.

FIG. 3G is a schematic illustration of an augmentation device and imaging system according to another embodiment of the invention.

FIG. 3H is a schematic illustration of an augmentation device and imaging system according to another embodiment of the invention.

FIG. 3I is a schematic illustration of an augmentation device and imaging system according to another embodiment of the invention.

FIG. 4 is a schematic illustration of a system for (MRI-)image-guided surgery according to an embodiment of the current invention.

FIG. 5 shows representational illustrations of three camera configurations according to different embodiments of the invention, a stereo camera arrangement (left), a single camera arrangement (center) and an omnidirectional camera arrangement (right).

FIG. 6A is a schematic illustration of an augmentation device for a handheld imaging system according to an embodiment including a switchable semi-transparent screen for projection purposes.

FIG. 6B is a schematic illustration of an augmentation device for a handheld imaging system according to another embodiment including a switchable semi-transparent screen for projection purposes.

FIG. 7 is a schematic illustration of an augmentation device for a handheld imaging system according to an embodiment including a laser-based system for photoacoustic imaging (utilizing both tissue- and airborne laser and ultrasound waves) for needle tracking and improved imaging quality in some applications.

FIG. 8A is a schematic illustration of one possible approach for needle guidance, using projected guidance information overlaid directly onto the imaged surface, with an intuitive dynamic symbol scheme for position/orientation correction support.

FIG. 8B is another schematic illustration of an approach for needle guidance, using projected guidance information overlaid directly onto the imaged surface, with an intuitive dynamic symbol scheme for position/orientation correction support.

FIG. 9 shows the appearance of a needle touching a surface in a structured light system for an example according to an embodiment of the current application.

FIG. 10 shows surface registration results using CPD on points acquired from CT and a ToF camera for an example according to an embodiment of the current application.

FIG. 11 shows a comparison of SNR and CNR values that show a large improvement in quality and reliability of strain calculation when the RF pairs are selected using our automatic frame selection method for an example according to an embodiment of the current application.

FIG. 12 shows a breast phantom imaged with a three-color sine wave pattern; right the corresponding 3D reconstruction for an example according to an embodiment of the current application.

FIG. 13 shows laparoscopic partial nephrectomy guided by US elasticity imaging for an example according to an embodiment of the current application. Left: System concept and overview. Right: Augmented visualization.

FIG. 14 shows laparoscopic partial nephrectomy guided by US probe placed outside the body for an example according to an embodiment of the current application.

FIG. 15 shows an example of a photoacoustic-based registration method according to an embodiment of the current application. The pulsed laser projector initiates a pattern that can generate PA signals in the US space. Hence, fusion of both US and Camera spaces can be easily established using point-to-point real-time registration method.

FIG. 16 shows ground truth (left image) reconstructed by the complete projection data according to an embodiment of the current application. The middle one is reconstructed using the truncated sonogram with 200 channels trimmed from both sides. The right one is constructed using the truncated data and the extracted trust region (Rectangle support).

FIG. 17 is a schematic illustration showing projection of live ultrasound (useful as structured-light pattern and for guidance) onto the skin surface.

FIG. 18 is a schematic illustration of different structured-light patterns shown with varying spatial frequencies.

FIG. 19 is a schematic illustration of different structured-light patterns, with and without edges, to aid the detection of straight needles.

FIG. 20 is a schematic illustration of randomizing through different patterns over time to increase the data density for stereo surface reconstruction.

FIG. 21 is a schematic illustration of use of a camera/projection unit combination outside of an imaging device next to the patient; here projecting structured-light patterns onto the skin as well as onto a semi-transparent or switchable-film screen above the patient.

FIG. 22 is a schematic illustration of using a switchable-film, fluorescent, or similar semi-transparent screen, simultaneous projection onto both the patient and the screen is possible.

FIG. 23 is a schematic illustration of dual-shadow passive guidance—by projecting one line from each projection center, two light planes are created that intersect at the desired needle pose and allow passive alignment.

FIG. 24 is a schematic illustration of semi-active, single-shadow guidance—by projecting one line and additional guidance symbols (based on needle tracking results), the needle can be passively aligned in one plane and actively in the remaining degrees of freedom.

FIG. 25 is a schematic illustration of using “bulby” (bottom) as opposed to straight lines (top) to improve needle guidance performance and usability because of the additional directional information to the user. Critical regions projected onto the surface, from the point of view of the projector, needle, or other viewpoints.

FIG. 26A is a schematic illustration of a setup for camera-ultrasound calibration with double-wedge phantom. The ultrasound probe becomes aligned with the wedges' central plane during a manual sweep, and simultaneously a stereo view of a grid allows to reconstruct the camera pose relative to the well-known phantom.

FIG. 26B is an illustration of a multi-line phantom. This figure shows another configuration of a known geometry that can uniquely identify the pose of the ultrasound imaging frame, and relate the ultrasound image to the known optical landmark (the checker board). Hence the calibration can be performed from a single image.

FIG. 27 is a schematic illustration of estimation of the camera pose in camera coordinates allows to optimize ultrasound imaging parameters (such as focus depth) for best needle or target imaging.

FIG. 28A is a schematic illustration of target/directional symbols indicate the changes to the needle pose to be made by the user in order to align with the target.

FIG. 28B is a schematic illustration of dual-shadow approach for passive guidance.

FIG. 28C is a schematic illustration of direct projection of target/critical regions onto the surface allows freehand navigation by the user.

FIG. 29 is a schematic illustration of projection of visible rays from the projection center onto arbitrary surfaces allows to reconstruct lines that in turn allow to reconstruct the projection center in camera coordinates, helping to calibrate cameras and projectors.

FIG. 30 is a schematic illustration of the system uses the projected insertion points as a “capture range” reference, discarding/not tracking needles that point too far away from it.

FIG. 31 is a schematic illustration of passive needle alignment using one projector, one camera: Alignment of the needle with the projected line constrains the pose to a plane, while alignment with a line overlaid onto the camera image imposes another plane; together defining a needle insertion pose.

FIG. 32 is a schematic illustration of double-shadow passive guidance with a single projector and dual-mirror attachment: The single projection cone is split into two virtual cones from different virtual centers, thus allowing passive alignment with limited hardware overhead.

FIG. 33 is a picture illustrating how double-wedges show up in ultrasound and how they are automatically detected/segmented (the green triangle). This is the pose recovery based on ultrasound images.

FIG. 34 is a screenshot of the system's graphical user interface showing the image overlay for out-of-plane views (the top section, with the green crosshair+line crossing the horizontal gray “ultrasound plane” line).

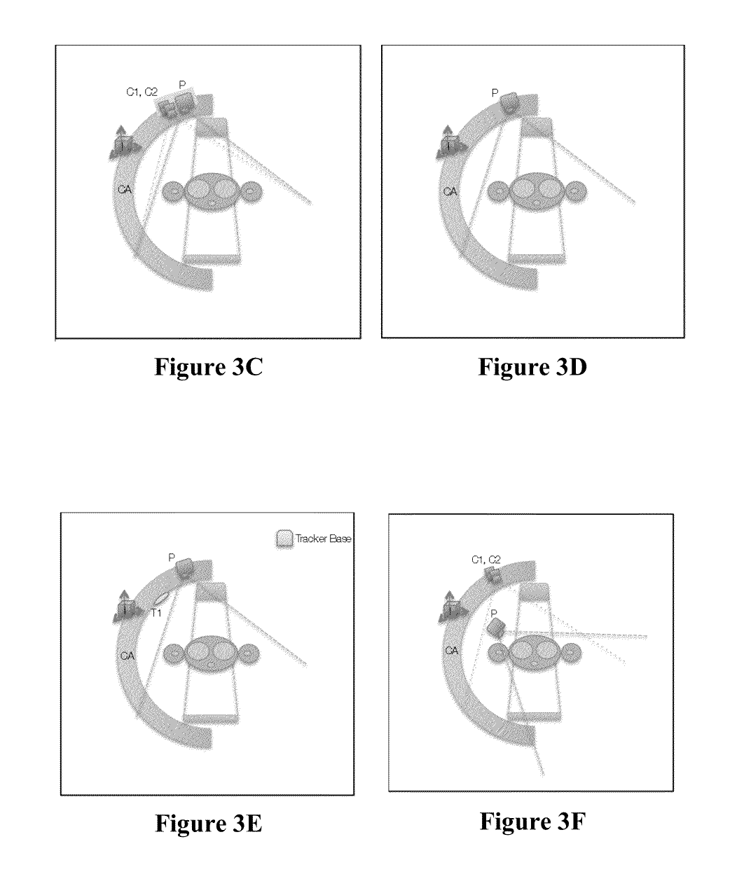

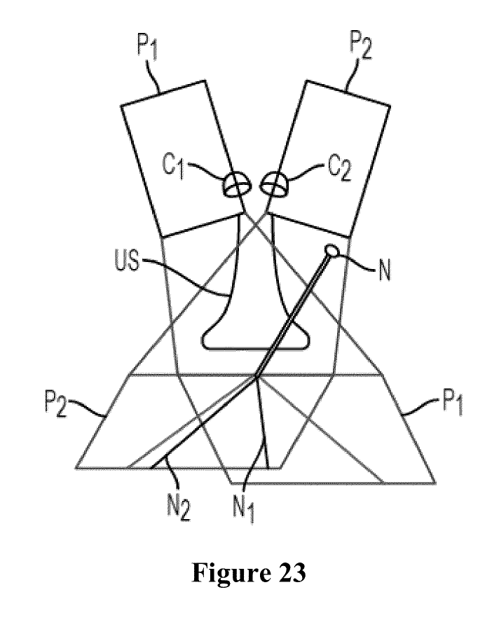

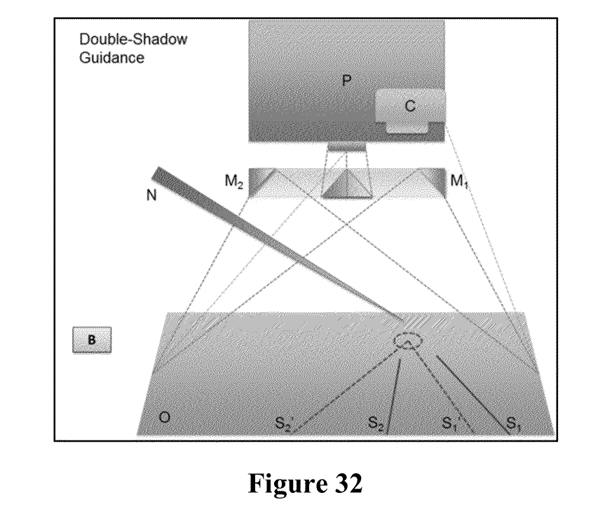

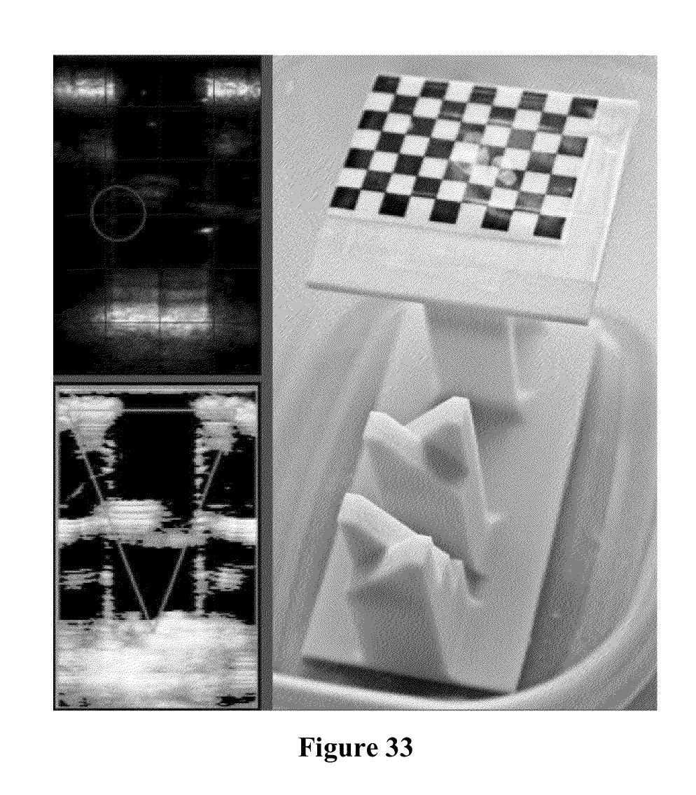

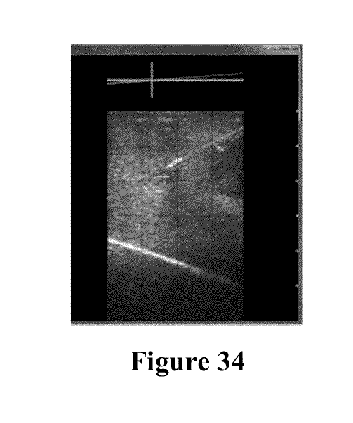

In FIGS. 5 and 17 through 32, projected images are shown in blue, and camera views are shown in red. Additionally, C denotes cameras 1&2; P is projector, P′ is projected image (blue); C′ is camera views (red); N is needle or instrument; M is mirror; B is base, US is ultrasound, I is imaging system, SLS is structured light surface, O is object or patient surface, and S is for a semi-transparent or switchable-film screen (except for FIGS. 24 and 32, where S is a real (cast) line shadow, and S′ are projected shadow lines for alignment).

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification are incorporated by reference as if each had been individually incorporated.

Some embodiments of this invention describe IGI-(image-guided interventions)-enabling “platform technology” going beyond the current paradigm of relatively narrow image-guidance and tracking. It simultaneously aims to overcome limitations of tracking, registration, visualization, and guidance; specifically using and integrating techniques e.g. related to needle identification and tracking using 3D computer vision, structured light, and photoacoustic effects; multi-modality registration with novel combinations of orthogonal imaging modalities; and imaging device tracking using local sensing approaches; among others.

The current invention covers a wide range of different embodiments, sharing a tightly integrated common core of components and methods used for general imaging, projection, vision, and local sensing.

Some embodiments of the current invention are directed to combining a group of complementary technologies to provide a local sensing approach that can provide enabling technology for the tracking of medical imaging devices, for example, with the potential to significantly reduce errors and increase positive patient outcomes. This approach can provide a platform technology for the tracking of ultrasound probes and other imaging devices, intervention guidance, and information visualization according to some embodiments of the current invention. By combining ultrasound imaging with image analysis algorithms, probe-mounted camera and projection units, and very low-cost, independent optical-inertial sensors, according to some embodiments of the current invention, it is possible to reconstruct the position and trajectory of the device and possible tools or other objects by incrementally tracking their current motion.

Some embodiments of the current invention allow the segmentation, tracking, and guidance of needles and other tools (using visual, ultrasound, and possibly other imaging and localization modalities), allowing for example the integration with the above-mentioned probe tracking capabilities into a complete tracked, image-guided intervention system.

The same set of sensors can enable interactive, in-place visualization using additional projection components. This visualization can include current or pre-operative imaging data or fused displays thereof, but also navigation information such as guidance overlays.

The same projection components can help in surface acquisition and multi-modality registration, capable of reliable and rapid fusion with pre-operative plans, in diverse systems such as handheld ultrasound probes, MRI/CT/C-arm imaging systems, wireless capsule endoscopy, and conventional endoscopic procedures, for example.

Such devices can allow imaging procedures with improved sensitivity and specificity as compared to the current state of the art. This can open up several possible application scenarios that previously required harmful X-ray/CT or expensive MRI imaging, and/or external tracking, and/or expensive, imprecise, time-consuming, or impractical hardware setups, or that were simply afflicted with an inherent lack of precision and guarantee of success, such as:
diagnostic imaging in cancer therapy, prenatal imaging etc.: can allow the generation of freehand three-dimensional ultrasound volumes without the need for external tracking,
biopsies, RF/HIFU ablations etc.: can allow 2D- or 3D-ultrasound-based needle guidance without external tracking,
brachytherapy: can allow 3D-ultrasound acquisition and needle guidance for precise brachytherapy seed placement,
cone-beam CT reconstruction: can enable high-quality C-arm CT reconstructions with reduced radiation dose and focused field of view,
gastroenterology: can perform localization and trajectory reconstruction for wireless capsule endoscopes over extended periods of time, and
other applications relying on tracked imaging and tracked tools.

Some embodiments of the current invention can provide several advantages over existing technologies, such as combinations of:
single-plane US-to-CT/MRI registration—no need for tedious acquisition of US volumes,
low-cost tracking—no optical or electro-magnetic (EM) tracking sensors on handheld imaging probes, tools, or needles, and no calibrations necessary,
in-place visualization—guidance information and imaging data is not displayed on a remote screen, but shown projected on the region of interest or over it onto a screen,
local, compact, and non intrusive solution—ideal tracking system for hand-held and compact ultrasound systems that are primarily used in intervention and point-of-care clinical suites, but also for general needle/tool tracking under visual tracking in other interventional settings,
improved quality of cone-beam CT—truncation artifacts are minimized.
improved tracking and multi-modality imaging for capsule endoscopes—enables localization and diagnosis of suspicious findings,
improved registration of percutaneous ultrasound and endoscopic video, using pulsed-laser photoacoustic imaging.

For example, some embodiments of the current invention are directed to devices and methods for the tracking of ultrasound probes and other imaging devices. By combining ultrasound imaging with image analysis algorithms, probe-mounted cameras, and very low-cost, independent optical-inertial sensors, it is possible to reconstruct the position and trajectory of the device and possible tools or other objects by incrementally tracking their current motion according to an embodiment of the current invention. This can provide several possible application scenarios that previously required expensive, imprecise, or impractical hardware setups. Examples can include the generation of freehand three-dimensional ultrasound volumes without the need for external tracking, 3D ultrasound-based needle guidance without external tracking, improved multi-modal registration, simplified image overlay, or localization and trajectory reconstruction for wireless capsule endoscopes over extended periods of time, for example.

The same set of sensors can enable interactive, in-place visualization using additional projection components according to some embodiments of the current invention.

Current sonographic procedures mostly use handheld 2D ultrasound (US) probes that return planar image slices through the scanned 3D volume (the “region of interest”/ROI). In this case, in order to gain sufficient understanding of the clinical situation, the sonographer needs to scan the ROI from many different positions and angles and mentally assemble a representation of the underlying 3D geometry. Providing a computer system with the sequence of 2D images together with the transformations between successive images (“path”) can serve to algorithmically perform this reconstruction of a complete 3D US volume. While this path can be provided by conventional optical, EM etc. tracking devices, a solution of substantially lower cost would hugely increase the use of 3D ultrasound.

For percutaneous interventions requiring needle guidance, prediction of the needle trajectory is currently based on tracking with sensors attached to the distal (external) needle end and on mental extrapolation of the trajectory, relying on the operator's experience. An integrated system with 3D ultrasound, needle tracking, needle trajectory prediction and interactive user guidance would be highly beneficial.

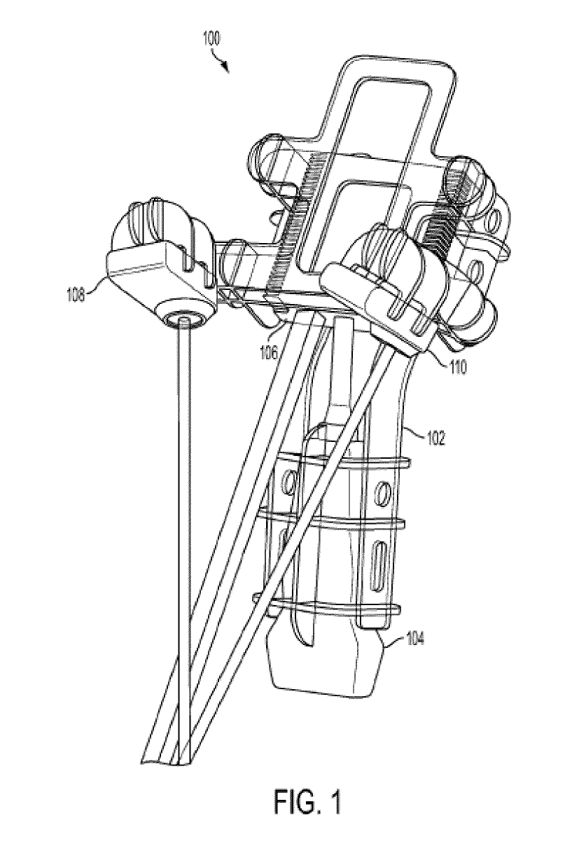

FIG. 1 is an illustration of an embodiment of an augmentation device 100 for an imaging system according to an embodiment of the current invention. The augmentation device 100 includes a bracket 102 that is structured to be attachable to an imaging component 104 of the imaging system. In the example of FIG. 1, the imaging component 104 is an ultrasound probe and the bracket 102 is structured to be attached to a probe handle of the ultrasound probe. However, the broad concepts of the current invention are not limited to only this example. The bracket 102 can be structured to be attachable to other handheld instruments for image-guided surgery, such as surgical orthopedic power tools or stand-alone handheld brackets, for example. In other embodiments, the bracket 102 can be structured to be attachable to the C-arm of an X-ray system or an MRI system, for example.

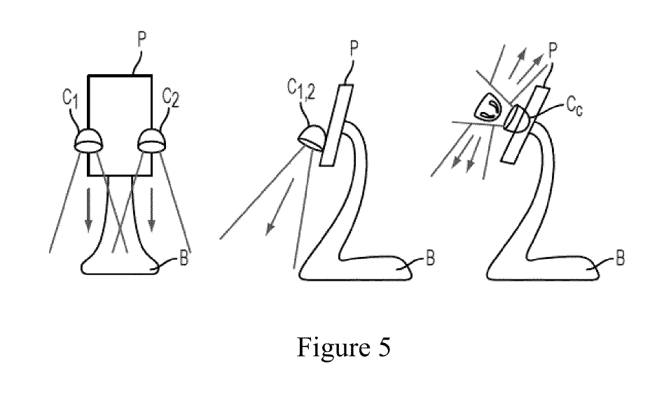

The augmentation device 100 also includes a projector 106 attached to the bracket 102. The projector 106 is arranged and configured to project an image onto a surface in conjunction with imaging by the imaging component 104. The projector 106 can be at least one of a visible light imaging projector, a laser imaging projector, a pulsed laser, or a projector of a fixed or selectable pattern (using visible, laser, or infrared/ultraviolet light). Depending on the application, the use of different spectral ranges and power intensities enables different capabilities, such as infrared for structured light illumination simultaneous with e.g. visible overlays; ultraviolet for UV-sensitive transparent glass screens (such as MediaGlass, SuperImaging Inc.); or pulsed laser for photoacoustic imaging, for example. A fixed pattern projector can include, for example, a light source arranged to project through a slide, a mask, a reticle, or some other light-patterning structure such that a predetermined pattern is projected onto the region of interest. This can be used, for example, for projecting structured light patterns (such as grids or locally unique patterns) onto the region of interest. Another use for such projectors can be the overlay of user guidance information onto the region of interest, such as dynamic needle-insertion-supporting symbols (circles and crosses, cf. FIG. 8). Such a projector can be made to be very compact in some applications. A projector of a selectable pattern can be similar to the fixed pattern device, but with a mechanism to select and/or exchange the light-patterning component. For example, a rotating component could be used in which one of a plurality of predetermined light-patterning sections is moved into the path of light from the light source to be projected onto the region of interest. In other embodiments, said projector(s) can be a stand-alone element of the system, or combined with a subset of other components described in the current invention, i.e. not necessarily integrated in one bracket or holder with another imaging device. In some embodiments, the projector(s) may be synchronized with the camera(s), imaging unit, and/or switchable film screens.

The augmentation device 100 can also include at least one of a camera 108 attached to the bracket 102. In some embodiments, a second camera 110 can also be attached to the bracket 102, either with or without the projector, to provide stereo vision, for example. The camera can be at least one of a visible-light camera, an infra-red camera, or a time-of-flight camera in some embodiments of the current invention. The camera(s) can be stand-alone or integrated with one or more projection units in one device as well, depending on the application. They may have to be synchronized with the projector(s) and/or switchable film glass screens as well.

Additional cameras and/or projectors could be provided—either physically attached to the main device, some other component, or free-standing—without departing from the general concepts of the current invention. The cameras need not be traditional perspective cameras, but maybe of other types such as catadioptric or other omni-direction designs, line scan, and so forth. See, e.g., FIG. 5.

The camera 108 and/or 110 can be arranged to observe a surface region close to the and during operation of the imaging component 104. In the embodiment of FIG. 1, the two cameras 108 and 110 can be arranged and configured for stereo observation of the region of interest. Alternatively, one of the cameras 108 and 110, or an additional camera, or two, or more, can be arranged to track the user face location during visualization to provide information regarding a viewing position of the user. This can permit, for example, the projection of information onto the region of interest in such a way that it takes into account the position of the viewer, e.g. to address the parallax problem.

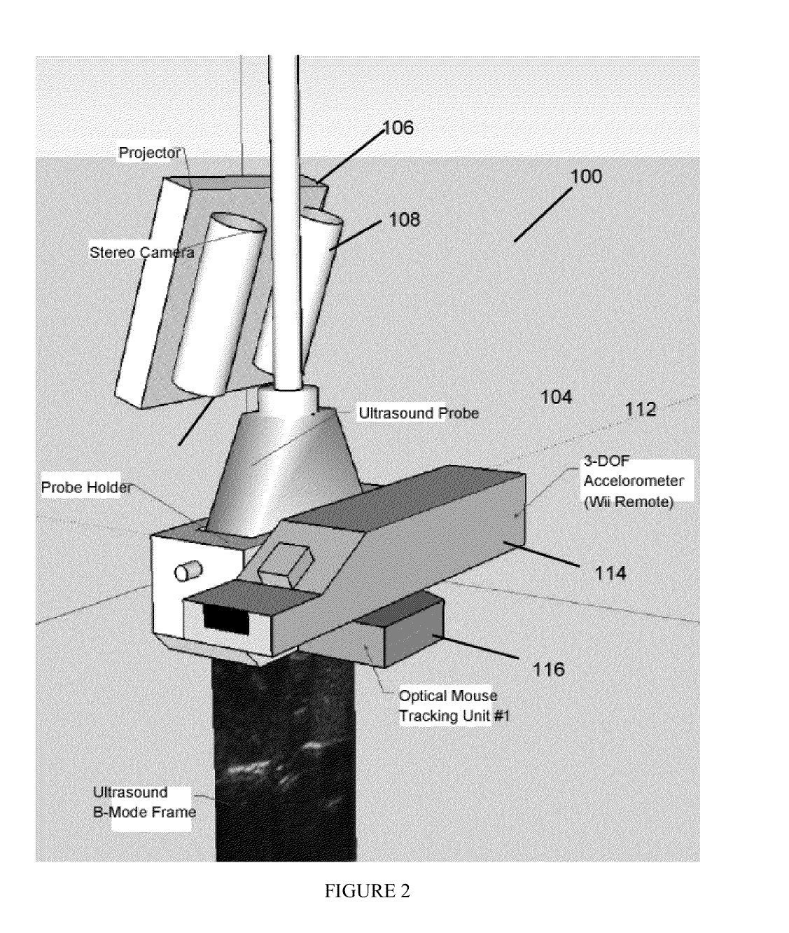

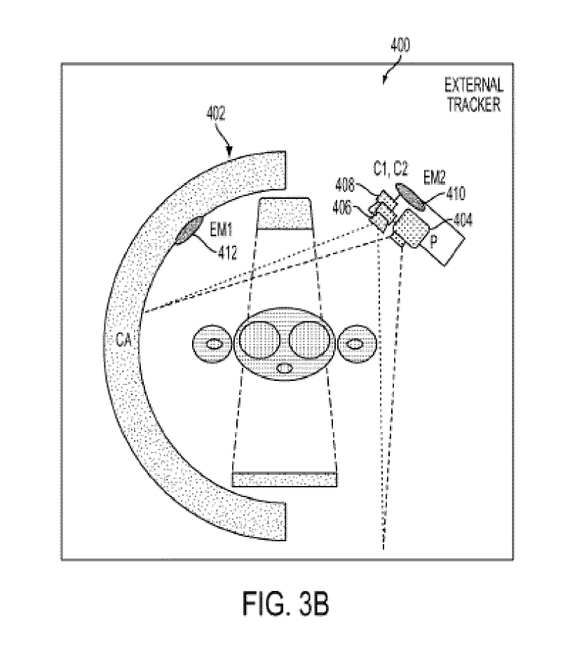

FIG. 2 is a schematic illustration of the augmentation device 100 of FIG. 1 in which the bracket 102 is not shown for clarity. FIG. 2 illustrates further optional local sensing components that can be included in the augmentation device 100 according to some embodiments of the current invention. For example, the augmentation device 100 can include a local sensor system 112 attached to the bracket 102. The local sensor system 112 can be part of a conventional tracking system, such as an EM tracking system, for example. Alternatively, the local sensor system 112 can provide position and/or orientation information of the imaging component 104 to permit tracking of the imaging component 104 while in use without the need for external reference frames such as with conventional optical or EM tracking systems. Such local sensor systems can also help in the tracking (e.g. determining the orientation) of handheld screens (FIG. 4) or capsule endoscopes, not just of imaging components. In some embodiments, the local sensor system 112 can include at least one of an optical, inertial, or capacitive sensor, for example. In some embodiments, the local sensor system 112 includes an inertial sensor component 114 which can include one or more gyroscopes and/or linear accelerometers, for example. In one embodiment, the local sensor system 112 has a three-axis gyro system that provides rotation information about three orthogonal axes of rotation. The three-axis gyro system can be a micro-electromechanical system (MEMS) three-axis gyro system, for example. The local sensor system 112 can alternatively, or in addition, include one or more linear accelerometers that provide acceleration information along one or more orthogonal axes in an embodiment of the current invention. The linear accelerometers can be, for example, MEMS accelerometers.

In addition to, or instead of the inertial sensor component 114, the local sensor system 112 can include an optical sensor system 116 arranged to detect motion of the imaging component 104 with respect to a surface. The optical sensor system 116 can be similar to the sensor system of a conventional optical mouse (using visible, IR, or laser light), for example. However, in other embodiments, the optical sensor system 116 can be optimized or otherwise customized for the particular application. This may include the use of (potentially stereo) cameras with specialized feature and device tracking algorithms (such as scale-invariant feature transform/SIFT and simultaneous localization and mapping/SLAM, respectively) to track the device, various surface features, or surface region patches over time, supporting a variety of capabilities such as trajectory reconstruction or stereo surface reconstruction.

In addition to, or instead of the inertial sensor component 114, the local sensor system 112 can include a local ultrasound sensor system to make use of the airborne photoacoustic effect. In this embodiment, one or more pulsed laser projectors direct laser energy towards the patient tissue surface, the surrounding area, or both, and airborne ultrasound receivers placed around the probe itself help to detect and localize potential objects such as tools or needles in the immediate vicinity of the device.

In some embodiments, the projector 106 can be arranged to project an image onto a local environment adjacent to the imaging component 104. For example, the projector 106 can be adapted to project a pattern onto a surface in view of the cameras 108 and 110 to facilitate stereo object recognition and tracking of objects in view of the cameras. For example, structured light can be projected onto the skin or an organ of a patient according to some embodiments of the current invention. According to some embodiments, the projector 106 can be configured to project an image that is based on ultrasound imaging data obtained from the ultrasound imaging device. In some embodiments, the projector 106 can be configured to project an image based on imaging data obtained from an x-ray computed tomography imaging device or a magnetic resonance imaging device, for example. Additionally, preoperative data or real-time guidance information could also be projected by the projector 106.

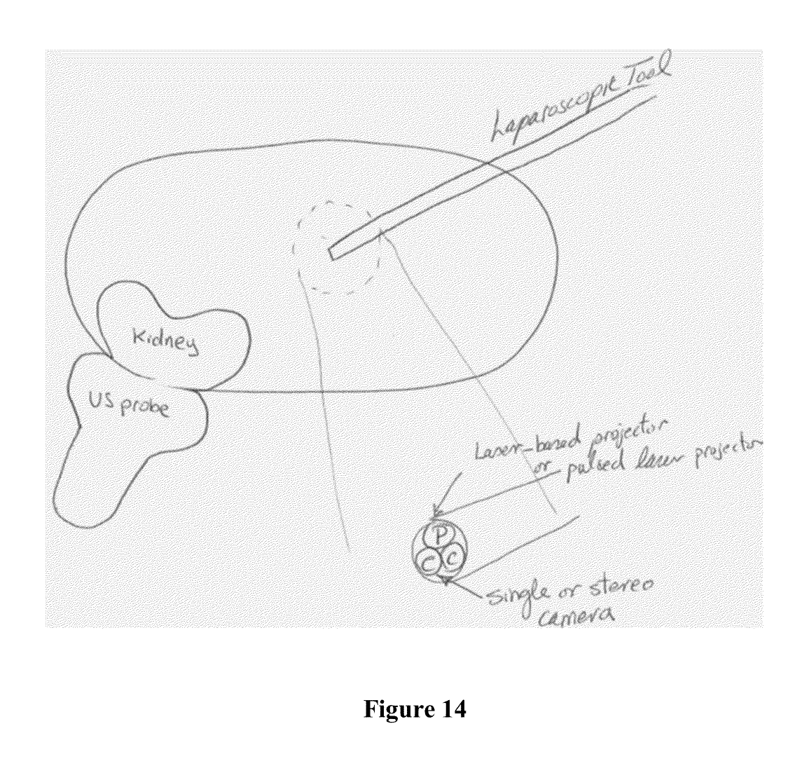

Although reconstruction using stereo vision is improved by projecting a pattern that aids in stereo matching performance, projecting a traditional structured light pattern may be distracting to the surgeon. However, the speckle pattern of an ultrasound image provides a natural form of texture that can also be informative to the surgeon. Thus, the invention may include the projection of the ultrasound data, and simultaneously that projection may be used to improve stereo reconstruction performance. See, e.g., FIG. 17.

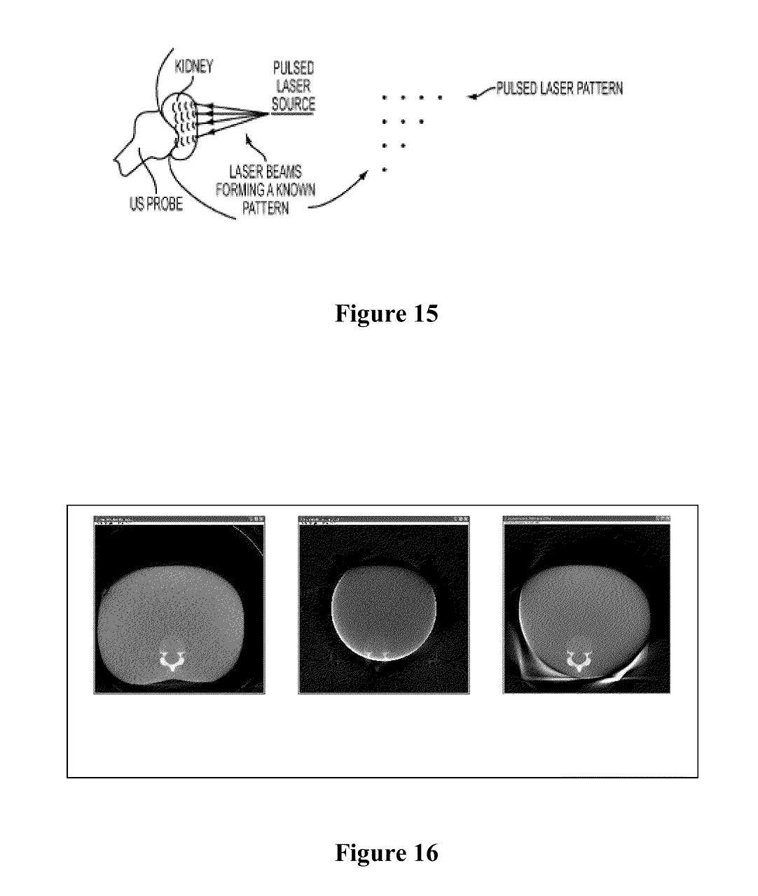

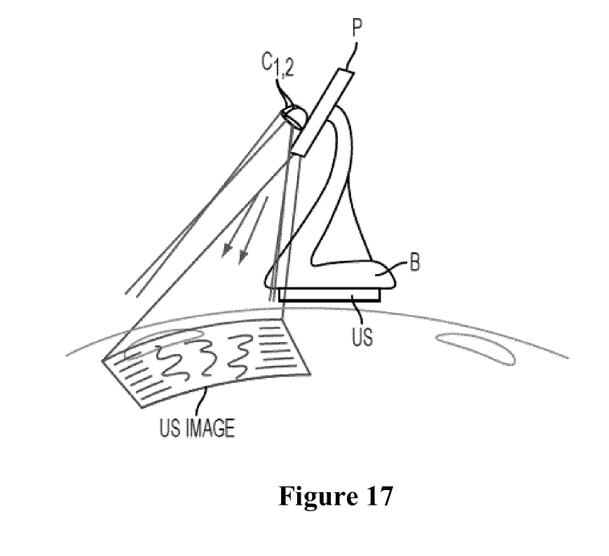

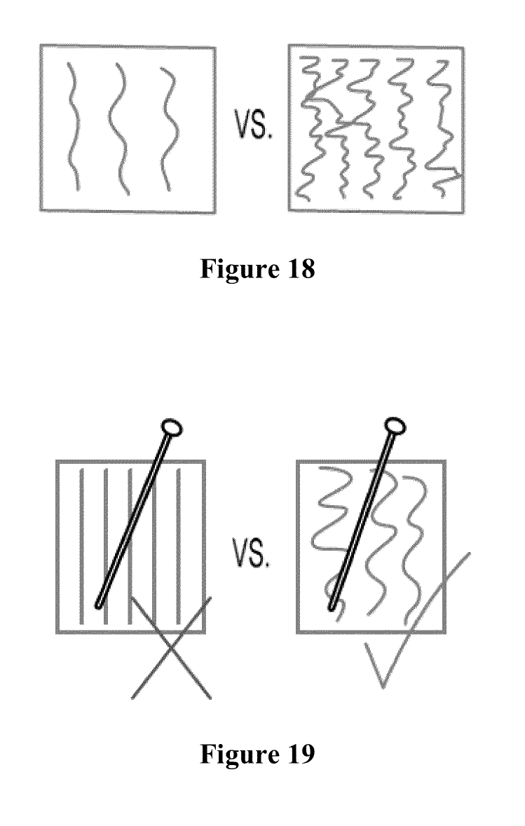

Alternatively, to improve stereo matching performance for surface reconstruction, it may prove useful to modify parameters of the projected pattern,—both within an image as well as over time. Such parameters may include (a) spatial frequencies (both the presence of edges vs. smoother transitions as well as color patch sizes)—to adapt to surface distance, apparent structure sizes, or camera resolutions, see, e.g., FIGS. 18 and 19,—or (b) colors—to adapt to surface properties such as skin type or environment conditions such as ambient lighting, or (c) to randomize/iterate through different patterns over time, see, e.g., FIG. 20. Both structured-light patterns as well as projected guidance symbols contribute to surface reconstruction performance, but can also be detrimental to overall system performance, e.g. when straight edges interfere with needle tracking. In such cases, projection patterns and guidance symbols can be adapted to optimize system metrics (such as tracking success/robustness, surface outlier ratio etc.), e.g. by introducing more curvy features.

The augmentation device 100 can also include a communication system that is in communication with at least one of the local sensor system 112, camera 108, camera 110 or projector 106 according to some embodiments of the current invention. The communication system can be a wireless communication system according to some embodiments, such as, but not limited to, a Bluetooth wireless communication system.

Although FIGS. 1 and 2 illustrate the imaging system as an ultrasound imaging system and that the bracket 102 is structured to be attached to an ultrasound probe handle 104, the broad concepts of the current invention are not limited to this example. The bracket can be structured to be attachable to other imaging systems, such as, but not limited to, x-ray and magnetic resonance imaging systems, for example.

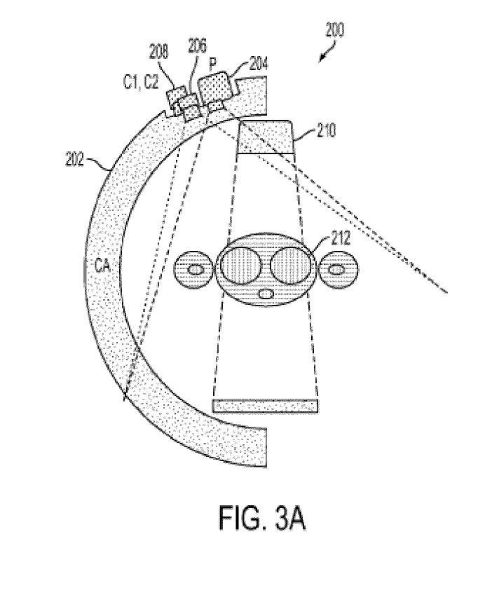

FIG. 3A is a schematic illustration of an augmentation device 200 attached to the C-arm 202 of an x-ray imaging system. In this example, the augmentation device 200 is illustrated as having a projector 204, a first camera 206 and a second camera 208. Conventional and/or local sensor systems can also be optionally included in the augmentation device 200, improving the localization of single C-arm X-ray images by enhancing C-arm angular encoder resolution and estimation robustness against structural deformation.

In operation, the x-ray source 210 typically projects an x-ray beam that is not wide enough to encompass the patient's body completely, resulting in severe truncation artifacts in the reconstruction of so-called cone beam CT (CBCT) image data. The camera 206 and/or camera 208 can provide information on the amount of extension of the patient beyond the beam width. This information can be gathered for each angle as the C-arm 202 is rotated around the patient 212 and be incorporated into the processing of the CBCT image to at least partially compensate for the limited beam width and reduce truncation artifacts In addition, conventional and/or local sensors can provide accurate data of the precise angle of illumination by the x-ray source, for example (more precise than potential C-arm encoders themselves, and potentially less susceptible to arm deformation under varying orientations). Other uses of the camera-projection combination units are surface-supported multi-modality registration, or visual needle or tool tracking, or guidance information overlay. One can see that the embodiment of FIG. 3A is very similar to the arrangement of an augmentation device for an MRI system.

FIG. 3B is a schematic illustration of a system for image-guided surgery 400 according to some embodiments of the current invention. The system for image-guided surgery 400 includes an imaging system 402, and a projector 404 configured to project an image onto a region of interest during imaging by the imaging system 402. The projector 404 can be arranged proximate the imaging system 402, as illustrated, or it could be attached to or integrated with the imaging system. In this case, the imaging system 402 is illustrated schematically as an x-ray imaging system. However, the invention is not limited to this particular example. As in the previous embodiments, the imaging system could also be an ultrasound imaging system or a magnetic resonance imaging system, for example. The projector 404 can be at least one of a white light imaging projector, a laser light imaging projector, a pulsed laser, or a projector of a fixed or selectable pattern, for example.

The system for image-guided surgery 400 can also include a camera 406 arranged to capture an image of a region of interest during imaging by the imaging system. A second camera 408 could also be included in some embodiments of the current invention. A third, fourth or even more cameras could also be included in some embodiments. The region of interest being observed by the imaging system 402 can be substantially the same as the region of interest being observed with the camera 406 and/or camera 408. The cameras 406 and 408 can be at least one of a visible-light camera, an infra-red camera or a time-of-flight camera, for example. Each of the cameras 406, 408, etc. can be arranged proximate the imaging system 402 or attached to or integrated with the imaging system 402.

The system for image-guided surgery 400 can also include one or more sensor systems, such as sensor systems 410 and 412, for example. In this example, the sensor systems 410 and 412 are part of a conventional EM sensor system. However, other conventional sensor systems such as optical tracking systems could be used instead of or in addition to the EM sensor systems illustrated. Alternatively, or in addition, one or more local sensor systems such as local sensor system 112 could also be included instead of sensor systems 410 and/or 412. The sensor systems 410 and/or 412 could be attached to any one of the imaging system 402, the projector 404, camera 406 or camera 408, for example. Each of the projector 404 and cameras 406 and 408 could be grouped together or separate and could be attached to or made integral with the imaging system 402, or arranged proximate the imaging system 402, for example.

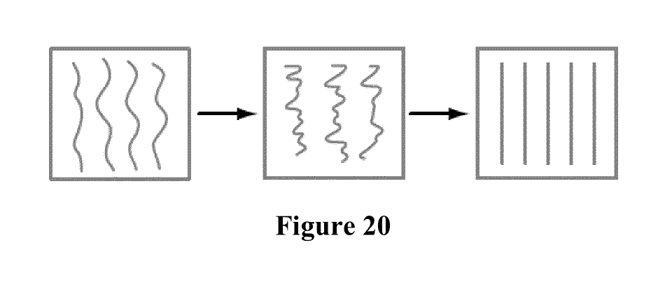

FIG. 4 illustrates one possible use of a camera/projection combination unit in conjunction with a medical imaging device such as MRI or CT. Image-guided interventions based on these modalities suffer from registration difficulties arising from the fact that in-place interventions are awkward or impossible due to space constraints within the imaging device bores, among other reasons. Therefore, a multi-modality image registration system supporting the interactive overlay of potentially fused pre- and intra-operative image data could support or enable e.g. needle-based percutaneous interventions with massively reduced imaging requirements in terms of duration, radiation exposure, cost etc. A camera/projection unit outside the main imaging system could track the patient, reconstruct the body surface using e.g. structured light and stereo reconstruction, and register and track needles and other tools relative to it. Furthermore, handheld units comprising switchable film glass screens could be tracked optically and used as interactive overlay projection surfaces, see, e.g., FIG. 21. The tracking accuracy for such screens could be improved by attaching (at least inertial) local sensor systems to said screens, allowing better orientation estimation that using visual clues alone. The screens need not impede the (potentially structured-light-supported) reconstruction of the underlying patient surface, nor block the user's view of that surface, as they can be rapidly switched (up to hundreds of times per second) alternating between a transparent mode to allow pattern and guidance information projection onto the surface, and an opaque mode to block and display other user-targeted data, e.g. in a tracked 3D data visualization fashion.

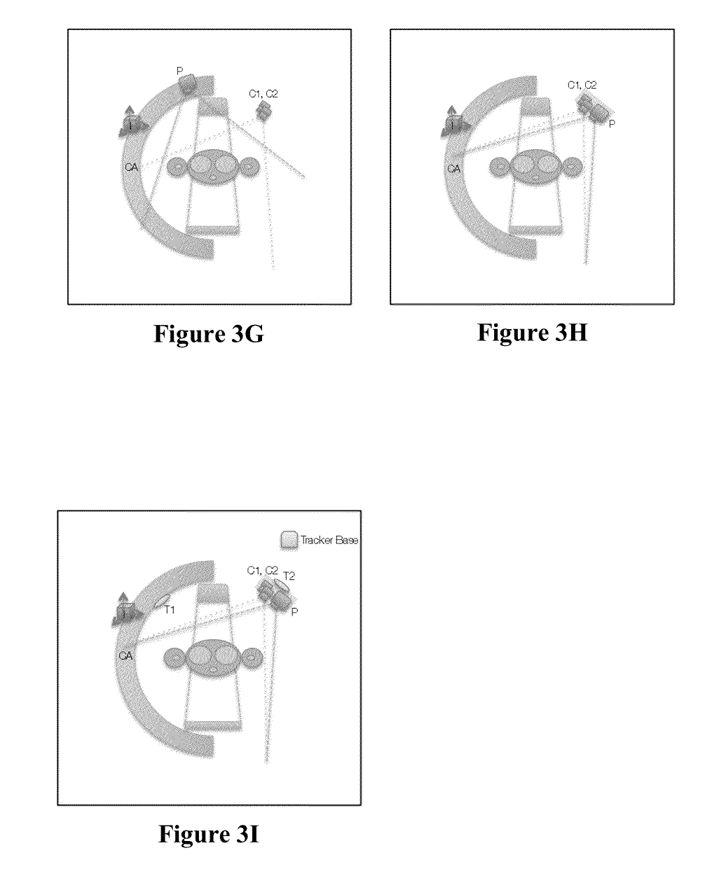

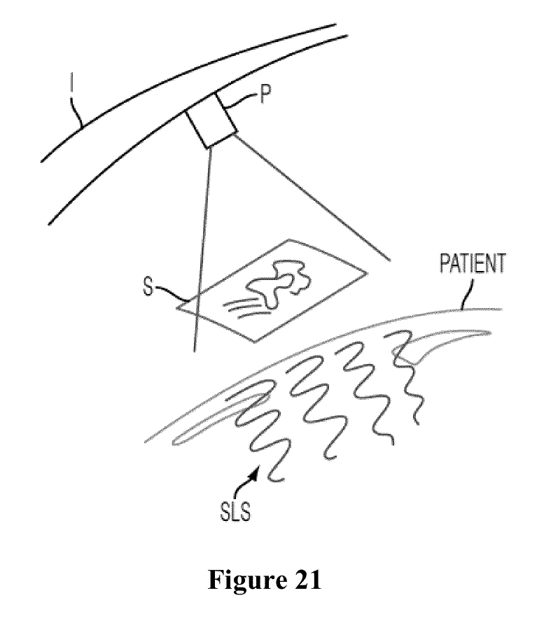

Such switchable film glass screens can also be attached to handheld imaging devices such as ultrasound probes and the afore-mentioned brackets as in FIG. 6. This way, imaging and/or guidance data can be displayed on a handheld screen—in opaque mode—directly adjacent to imaging devices in the region of interest, instead of on a remote monitor screen. Furthermore—in transparent mode—structured light projection and/or surface reconstruction are not impeded by the screen, see, e.g., FIG. 22. In both cases the data is projected onto or through the switchable screen using the afore-mentioned projection units, allowing a more compact handheld design or even remote projection. Furthermore, these screens (handheld or bracket-mounted) can also be realized using e.g. UV-sensitive/fluorescent glass, requiring a (potentially multi-spectral for color reproduction) UV projector to create bright images on the screen, but making active control of screen mode switching unnecessary. In the latter case, overlay data projection onto the screen and structured light projection onto the patient surface can be run in parallel, provided the structured light uses a frequency unimpeded by the glass.

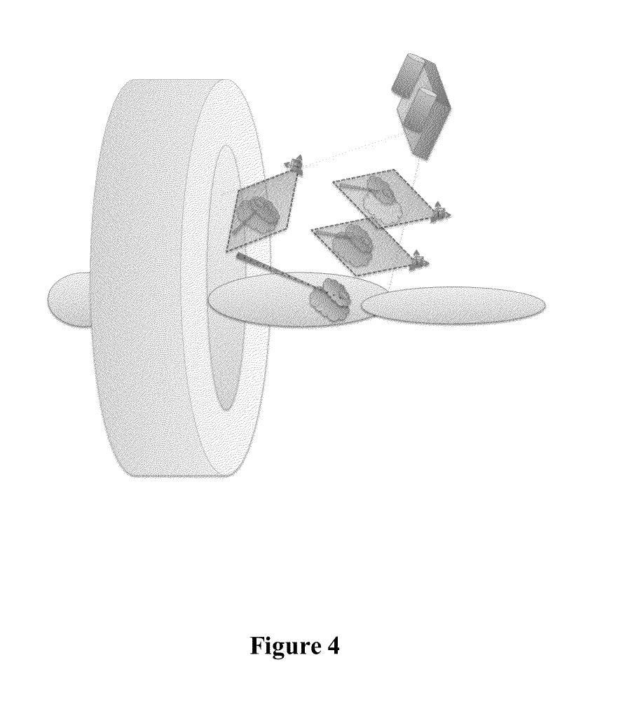

FIG. 7 describes a possible extension to the augmentation device (“bracket”) described for handheld imaging devices, comprising one or more pulsed lasers as projection units that are directed through fibers towards the patient surface, exciting tissue-borne photoacoustic effects, and towards the sides of the imaging device, emitting the laser pulse into the environment, allowing airborne photoacoustic imaging. For the latter, the handheld imaging device and/or the augmentation device comprise ultrasound receivers around the device itself, pointing into the environment. Both photoacoustic channels can be used e.g. to enable in-body and out-of-body tool tracking or out-of-plane needle detection and tracking, improving both detectability and visibility of tools/needles under various circumstances.

In endoscopic systems the photoacoustic effect can be used together with its structured-light aspect for registration between endoscopic video and ultrasound. By emitting pulsed laser patterns from a projection unit in an endoscopic setup, a unique pattern of light incidence locations is generated on the endoscope-facing surface side of observed organs. One or more camera units next to the projection unit in the endoscopic device observe the pattern, potentially reconstructing its three-dimensional shape on the organ surface. At the same time, a distant ultrasound imaging device on the opposite side of the organ under observation receives the resulting photoacoustic wave patterns and is able to reconstruct and localize their origins, corresponding to the pulsed-laser incidence locations. This “rear-projection” scheme allows simple registration between both sides—endoscope and ultrasound—of the system.

FIG. 8 outlines one possible approach to display needle guidance information to the user by means of direct projection onto the surface in the region of interest in a parallax-independent fashion, so the user position is not relevant to the method's success (the same method can be applied to projection e.g. onto a device-affixed screen as described above, or onto handheld screens). Using e.g. a combination of moving, potentially color/size/thickness/etc.—coded circles and crosses, the five degrees of freedom governing a needle insertion (two each for insertion point location and needle orientation, and one for insertion depth and/or target distance) can be intuitively displayed to the user. In one possible implementation, the position and color of a projected circle on the surface indicate the intersection of the line between the current needle position and the target location with the patient surface, and said intersection point's distance from a planned insertion point. The position, color, and size of a projected cross can encode the current orientation of the needle with respect to the correct orientation towards the target location, as well as the needle's distance from the target. The orientation deviation is also indicated by an arrow pointing towards the proper position/orientation configuration. In another implementation, guidance information necessary to adjust the needle orientation can be projected as a virtual shadow onto the surface next to the needle insertion point, prompting the user to minimize the shadow length to properly orient the needle for insertion.

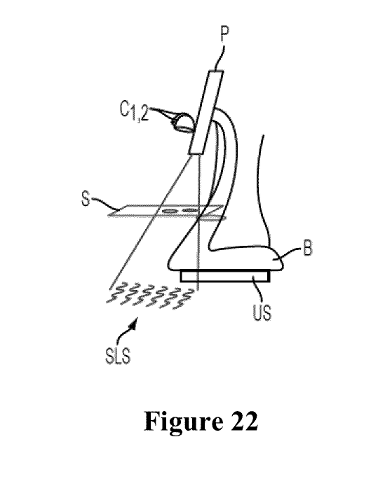

Needle guidance may be active, by projecting crosshairs or other targeting information for all degrees of freedom as described above. Needle guidance may also make use of shadows as a means of alignment. A “single-shadow alignment” can be used for 1 degree of freedom with additional active tracking/guidance for remaining degree of freedom, e.g. circles or crosshairs, see, e.g., FIG. 24. Alternatively, if multiple projectors are available, then stereo guidance may make use of shadows, active light planes, or other similar methods, see, e.g., FIGS. 23 and 32. In this case, needle guidance may be passive (without needle tracking) by using simple alignment either in stereo views/cameras or in dual projector shadows or patterns.

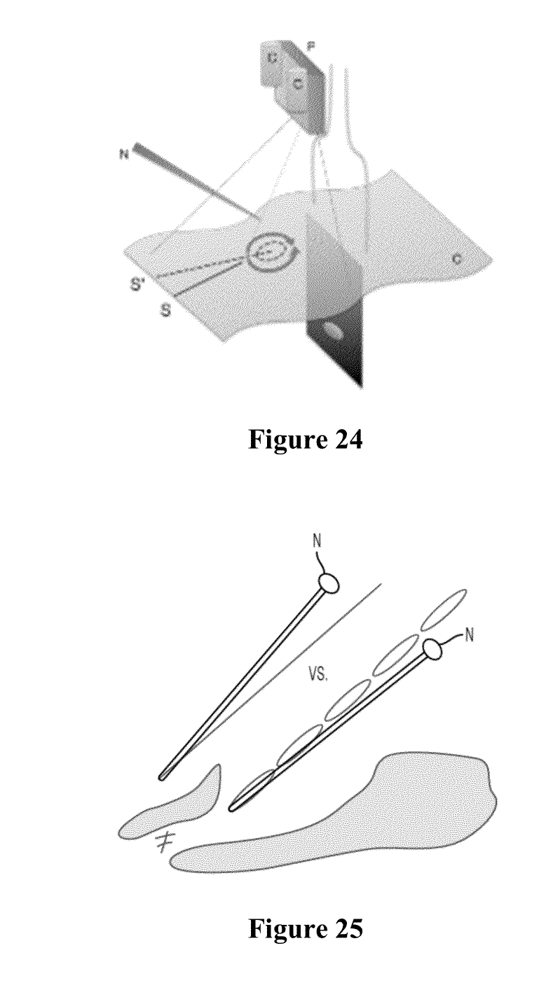

Specific projection patterns may be used to enhance the speed or reliability of tracking. Examples include specific shadow “brush types” or profiles to help quickly and precisely aligning needle shadow with projected shadow (“bulby lines” etc.). See, e.g., FIG. 25. Other patterns may be better for rough vs. precise alignments.

The system may also make use of “shadows” or projections of critical areas or forbidden regions onto patient surface, using pre-op CT/MRI or non-patient-specific atlas to define a “roadmap” for an intervention, see, e.g., FIG. 25.

While the above-mentioned user guidance display is independent of the user viewing direction, several other information displays (such as some variations on the image-guided intervention system shown in FIG. 4) may benefit from knowledge about the location of the user's eyes relative to the imaging device, the augmentation device, another handheld camera/projection unit, and/or projection screens or the patient surface. Such information can be gathered using one or more optical (e.g. visible- or infrared-light) cameras pointing away from the imaging region of interest towards regions of space where the user face may be expected (such as upwards from a handheld ultrasound imaging device) combined with face-detection capabilities to determine the user's eye location, for example.

### EXAMPLES

The following provides some examples according to some embodiments of the current invention. These examples are provided to facilitate a description of some of the concepts of the invention and are not intended to limit the broad concepts of the invention.

The local sensor system can include inertial sensors 506, such as a three-axis gyro system, for example. For example, the local sensor system 504 can include a three-axis MEMS gyro system. In some embodiments, the local sensor system 504 can include optical position sensors 508, 510 to detect motion of the capsule imaging device 500. The local sensor system 504 can permit the capsule imaging device 500 to record position information along with imaging data to facilitate registering image data with specific portions of a patient's anatomy after recovery of the capsule imaging device 500, for example.

Some embodiments of the current invention can provide an augmentation of existing devices which comprises a combination of different sensors: an inertial measurement unit based on a 3-axis accelerometer; one or two optical displacement tracking units (OTUs) for lateral surface displacement measurement; one, two or more optical video cameras; and a (possibly handheld and/or linear) ultrasound (US) probe, for example. The latter may be replaced or accompanied by a photoacoustic (PA) arrangement, i.e. one or more active lasers, a photoacoustically active extension, and possibly one or more separate US receiver arrays. Furthermore, an embodiment of the current invention may include a miniature projection device capable of projecting at least two distinct features.

These sensors (or a combination thereof) may be mounted, e.g. on a common bracket or holder, onto the handheld US probe, with the OTUs pointing towards and close to the scanning surface (if more than one, then preferably at opposite sides of the US array), the cameras mounted (e.g., in a stereo arrangement) so they can capture the environment of the scanning area, possible needles or tools, and/or the operating room environment, and the accelerometer in a basically arbitrary but fixed location on the common holder. In a particular embodiment, the projection device may be pointing mainly onto the scanning surface. In another particular embodiment, one PA laser may point towards the PA extension, while the same or another laser may point outwards, with US receiver arrays suitably arranged to capture possible reflected US echos. Different combinations of the mentioned sensors are possible.

The mounting bracket need not be limited to a fixed position or orientation. The augmentation device may be mounted on a re-configurable/rotatable setup to re-orient device from in-plane to out-of-plane projection and guidance depending on the needs of the operator. The mounting mechanism may also be configurable to allow elevation of augmentation device to accommodate different user habits (low/high needle grips etc.). The mounting system may also be modular and allow users to add cameras, add projectors, add mechanical guides e.g. for elevation angle control as needed for the application.

For particular applications and/or embodiments, an interstitial needle or other tool may be used. The needle or tool may have markers attached for better optical visibility outside the patient body. Furthermore, the needle or tool may be optimized for good ultrasound visibility if they are supposed to be inserted into the body. In particular embodiments the needle or tool may be combined with inertial tracking components (i.e. accelerometers).

For particular applications and/or embodiments, additional markers may optionally be used for the definition of registration or reference positions on the patient body surface. These may be optically distinct spots or arrangements of geometrical features designed for visibility and optimized optical feature extraction.

For particular applications and/or embodiments, the device to be augmented by the proposed invention may be a handheld US probe; for others it may be a wireless capsule endoscope (WCE); and other devices are possible for suitably defined applications, where said applications may benefit from the added tracking and navigational capabilities of the proposed invention.

### Software Components:

In one embodiment (handheld US probe tracking), an embodiment of the invention includes a software system for opto-inertial probe tracking (OIT). The OTUs generate local translation data across the scan surface (e.g. skin or intestinal wall), while accelerometers and/or gyroscopes provide absolute orientation and/or rotation motion data. Their streams of local data are combined over time to reconstruct an n-DoF probe trajectory with n=2 . . . 6, depending on the actual OIC sensor combination and the current pose/motion of the probe.

In general, the current pose Q(t)=(P(t), R(t)) can be computed incrementally with

$$P ( t ) = P ( 0 ) + {\sum }_{i = 0}^{t - 1} R ( i ) Δ p ( i )$$

where the R(i) are the orientations directly sampled from the accelerometers and/or incrementally tracked from relative displacements between the OTUs (if more than one) at time i, and Δp(i) are the lateral displacements at time i as measured by the OTUs. P(0) is an arbitrarily chosen initial reference position.

In one embodiment (handheld US probe tracking), a software system for speckle-based probe tracking is included. An (ultrasound-image-based) speckle decorrelation analysis (SDA) algorithm provides very high-precision 1-DoF translation (distance) information for single ultrasound image patch pairs by decorrelation, and 6-DoF information for the complete ultrasound image when combined with planar 2D-2D registration techniques. Suitable image patch pairs are preselected by means of FDS (fully developed speckle) detection. Precision of distance estimation is improved by basing the statistics on a larger set of input pairs.

Both approaches (opto-inertial tracking and SDA) may be combined to achieve greater efficiency and/or robustness. This can be achieved by dropping the FDS detection step in the SDA and instead relying on opto-inertial tracking to constrain the set of patch pairs to be considered, thus implicitly increasing the ratio of suitable FDS patches without explicit FDS classification.

Another approach can be the integration of opto-inertial tracking information into a maximum-a-posteriori (MAP) displacement estimation. In yet another approach, sensor data fusion between OTT and SDA can be performed using a Kalman filter.

In one embodiment (handheld US probe tracking), a software system for camera-based probe tracking and needle and/or tool tracking and calibration can be included.

The holder-mounted camera(s) can detect and segment e.g. a needle in the vicinity of the system. By detecting two points P₁ and P₂, with P₁ being the needle insertion point into the patient tissue (or alternatively, the surface intersection point in a water container) and P₂ being the end or another suitably distant point on the needle, and a third point Pᵢ being the needle intersection point in the US image frame, it is possible to calibrate the camera-US probe system in one step in closed form by following

(P₂−P₁)×(P₁−XPᵢ)=0

with X being the sought calibration matrix linking US frame and the camera(s).

Another method for calibrating an ultrasound device, a pair of cameras, and a projection device proceeds as follows. The projector projects a pattern onto a planar target. The planar target is observed by the cameras, and is simultaneously measured by the ultrasound probe. Several such images are acquired. Features on the planar target are used to produce a calibration for the camera system. Using this calibration, the position of the plane in space can be calculated by the camera system. The projector can be calibrated using the same information. The corresponding position of the intersection of the ultrasound beam with the plane produces a line in the ultrasound image. Processing of several such lines allows the computation of the relative position of the cameras and the ultrasound probe.

In order to insure high accuracy, synchronization of the imaging components is necessary. Synchronizing one or more cameras with an ultrasound system can be accomplished whereby a trigger signal is derived from or generated by the ultrasound system, and this trigger signal is use to trigger camera acquisition. The trigger signal may come from the ultrasound data acquisition hardware, or from the video display associated with the ultrasound system. The same trigger signal may be used to trigger a projection device to show a particular image or pattern.

An alternative is a method of software temporal synchronization whereby the camera pair and ultrasound system are moved periodically above a target. The motion of the target in both camera and ultrasound is measured, and the temporal difference is computed by matching or fitting the two trajectories. A method for doing so is disclosed in N. Padoy, G. D. Hager, Spatio-Temporal Registration of Multiple Trajectories, Proceedings of Medical Image Computing and Computer-Assisted Intervention (MICCAI), Toronto, Canada, September 2011.

This also provides a means for interleaving patterns for guidance and for other purposes such as stereo reconstruction, whereby a trigger signal causes the projector to switch between patterns. Preferentially, the pattern used by the camera system is invisible to the naked eye so that the user is not distracted by the transition.

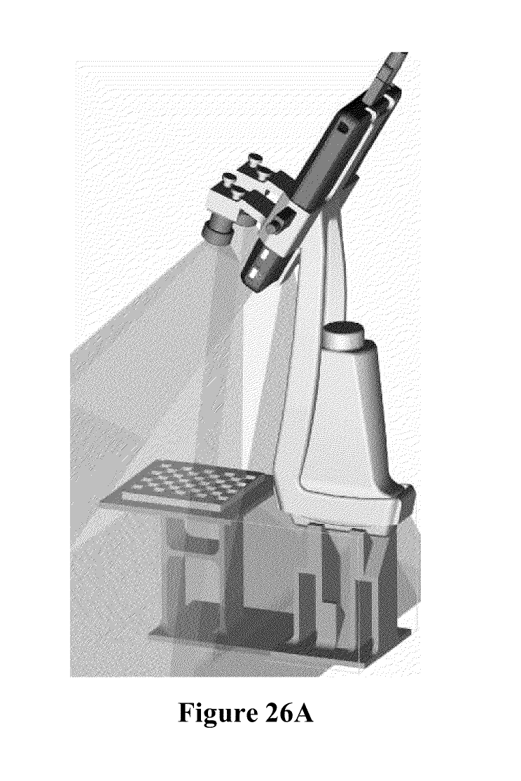

Calibration can also be accomplished by using a specially constructed volume, as shown in FIGS. 26A and 26B. The ultrasound system is swept over the volume while the volume is simultaneously observed by the camera system. The surface models from both ultrasound and the camera system are registered to a computational model of the shape, and from this the relative position of the camera and ultrasound system is computed.

An alternative implementation is to use nanocapsules that rupture under ultrasound irradiation, creating an opaque layer in a disposable calibration phantom

Furthermore, if the above-mentioned calibration condition does not hold at some point in time (detectable by the camera(s)), needle bending can be inferred from a single 2D US image frame and the operator properly notified.

Furthermore, 3D image data registration is also aided by the camera(s) overlooking the patient skin surface. Even under adverse geometrical conditions, three degrees of freedom (tilt, roll, and height) can be constrained using the cameras, facilitating registration of 3D US and e.g. CT or similar modalities by restricting the registration search space (making it faster) or providing initial transformation estimates (making it easier and/or more reliable). This may be facilitated by the application of optical markers onto the patient skin surface, which will also help in the creation of an explicit fixed reference coordinate system for integration of multiple 3D volumes.

Alternatively, drapes may be used that are designed to specifically enhance the performance of the system, whereby such drapes contain an easily detected pattern, fiducials, or other reference points, and the drapes adhere to the patient. Also, drapes that are transparent, and allow the cameras to see the patient directly through the drapes. Drapes may be specially colored to differentiate them from needles to be tracked. The drapes are preferably configured to enhance the ability of the cameras to compute probe motion.

Sterility can be preserved by using sterile probe coverings that contain special transparent areas for the cameras and projector to preserve sterility while also preserving or enhancing the function of the cameras and projectors.

In some embodiments, it may be useful to make use of pressure-sensitive drapes to indicate tissue deformation under the US probe. For example, such drapes could be used to enhance ultrasound elasticity measurement. The pressure-sensitive drapes may be used to monitor the use of the device by noting the level of pressure applied and correcting the registration and display based on that information.

Furthermore, the camera(s) provide additional data for pose tracking. In general, this will consist of redundant rotational motion information in addition to opto-inertial tracking. In special cases however, this information could not be recovered from OTT (e.g. yaw motions on a horizontal plane in case of surface tracking loss of one or both optical translation detectors, or tilt motion without translational components around a vertical axis). This information may originate from a general optical-flow-based rotation estimation, or specifically from tracking of specially applied optical markers onto the patient skin surface, which will also help in the creation of an explicit fixed reference coordinate system for integration of multiple 3D volumes.

Furthermore, by detecting and segmenting the extracorporeal parts of a needle, the camera(s) can provide needle translation information. This can serve as input for ultrasound elasticity imaging algorithms to constrain the search space (in direction and magnitude) for the displacement estimation step by tracking the needle and transforming estimated needle motion into expected motion components in the US frame, using the aforementioned calibration matrix X.

Furthermore, the camera(s) can provide dense textured 3D image data of the needle insertion area. This can be used to provide enhanced visualization to the operator, e.g. as a view of the insertion trajectory as projected down along the needle shaft towards the skin surface, using actual needle/patient images.

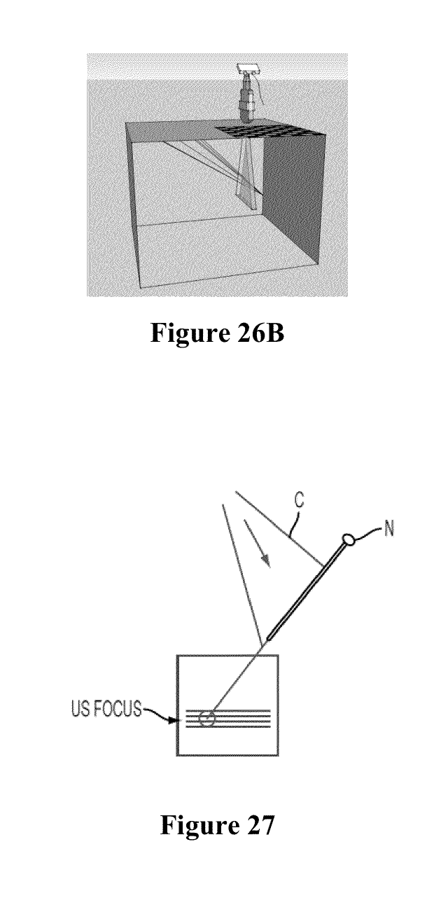

The system may use the pose (location and orientation) of the needle in air to optimize ultrasound to detect the needle in the body and vice-versa, see, e.g., FIG. 27.

It may be of interest to have differing fields of view and depth ranges in the depth imaging system. For example, on the surface, the cameras maybe a few 10s of centimeters from the surface; but at other times nearly a meter. In this case, it may be useful to have multiple depth ranging configurations built into the same head, mount, or bracket assembly), e.g. using three or four video cameras or multiple depth sensors, additionally at different relative orientations and/or set to different focal lengths.

For particular applications and/or embodiments, integration of a micro-projector unit can provide an additional, real-time, interactive visual user interface e.g. for guidance purposes. Projecting navigation data onto the patient skin in the vicinity of the probe, the operator need not take his eyes away from the intervention site to properly target subsurface regions. Tracking the needle using the aforementioned camera(s), the projected needle entry point (intersection of patient skin surface and extension of the needle shaft) given the current needle position and orientation can be projected using a suitable representation (e.g. a red dot). Furthermore, an optimal needle entry point given the current needle position and orientation can be projected onto the patient skin surface using a suitable representation (e.g. a green dot). These can be positioned in real-time, allowing interactive repositioning of the needle before skin puncture without the need for external tracking.

As noted previously, guidance can be visually provided to the user in a variety of ways, either (a) on-screen or (b) projected through one or more projectors, e.g. directly onto the patient surface near the probe.

Also, this guidance can be provided either (a) separately or (b) as an overlay to a secondary image stream, such as ultrasound images or mono- or multi-ocular camera views. Also, this guidance can be either (a) registered to the underlying image or environment geometry such that overlaid symbols correspond to environment features (such as target areas) in location and possibly size and/or shape, or (b) location-independent such that symbol properties, e.g. location, color, size, shape, but also auditory cues such as audio volume, sound clips, and/or frequency changes indicate to the user where to direct the tools or the probe.

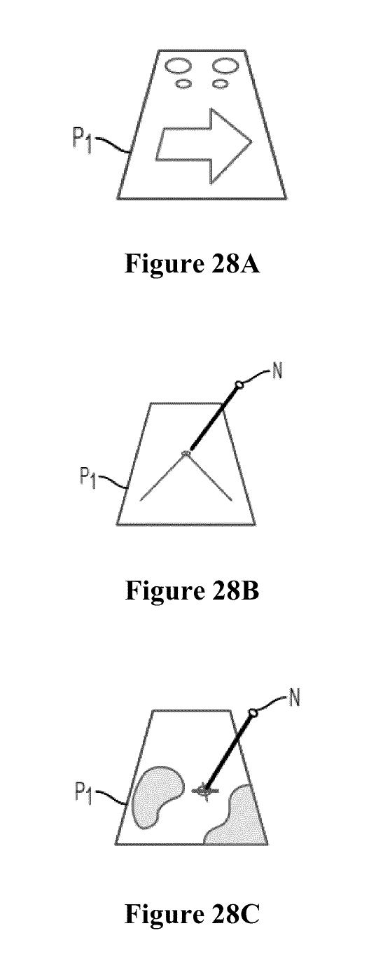

Guidance symbols can include—in order of increasing specificity—(a) proximity markers (to indicate general “closeness” by e.g. color-changing backgrounds, frames, or image tints, or auditory cues), (b) target markers (to point towards e.g. crosshairs, circles, bulls-eyes etc.), see, e.g., FIG. 28A, (c) alignment markers (to line up with e.g. lines, fans, polygons), see, e.g., FIG. 28B, or (d) area demarcations (to avoid e.g. shapes denoting critical regions, geometrically or anatomically inaccessible regions etc.), see, e.g., FIG. 28C.

Overlaid guidance symbols can interfere with overall system performance, e.g. when tracking needles; so adaptation of projected graphic primitives (such as replacing lines with elliptic or curvy structures) can reduce artifacts. Additionally, guidance “lines” composed of e.g. “string-of-pearls” series of circles/discs/ellipses etc. can improve alignment performance for the user. Additionally, the apparent thickness of guidance lines/structures can be modified based on detected tool width, distance to projector, distance to surface, excessive intervention duration, etc., to improve alignment performance.

Specific—non-exhaustive—examples of the above concepts include: a) overlaying crosshairs and/or extrapolated needle pose lines onto live ultrasound views on-screen or projected onto the patient; b) projecting paired symbols (circles, triangles etc.) that change size, color, and relative position depending on the current targeting error vector; c) overlaying alignment lines onto single/stereo/multiple camera views that denote desired needle poses, allowing the user to line up the camera image of the needle with the target pose, as well as lines denoting the currently-tracked needle pose for quality control purposes; and d) projecting needle alignment lines onto the surface, denoting both target pose (for guidance) as well as currently-tracked pose (for quality control), from one or more projectors.

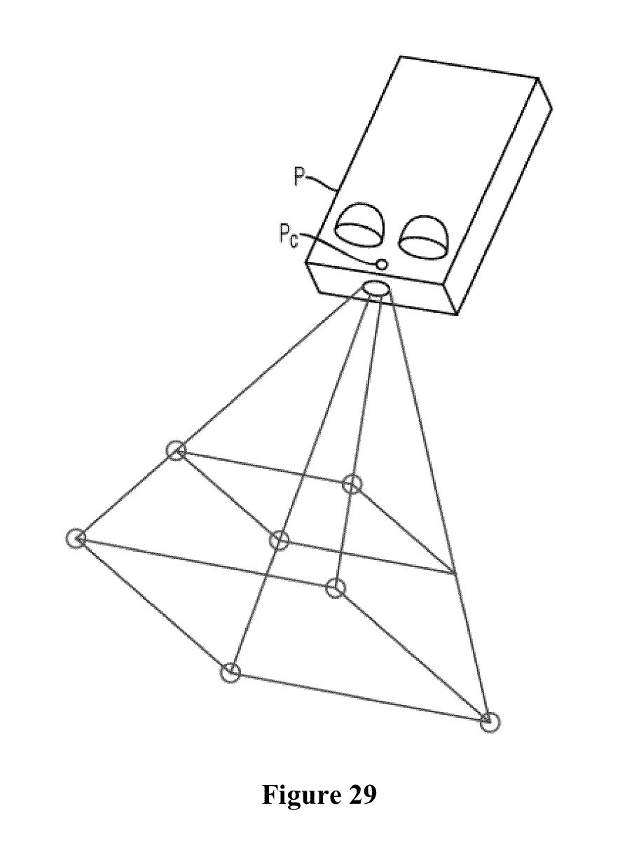

An important aspect of this system is a high accuracy estimate of the location of the projector relative to the probe and to the video camera. One means of doing so is to observe that visible rays projected from the camera will form straight lines in space that intersect at the optical center of the projector. Thus, with stereo cameras or a similar imaging system observing several surfaces upon which these rays fall, the system can calculate a series of 3D points which can then be extrapolated to compute the center of projection. See, e.g., FIG. 29. This can be performed with nearly any planar or nonplanar series of projection surfaces.

Different combinations of software components are possible for different applications and/or different hardware embodiments. Also, the overall configuration may be augmented by and/or controlled from a hand-held device such as a tablet computer for 1) ultrasound machine operation, 2) for visualization; 3) in addition, by using an one or more cameras on the tablet computer, for registration to patient for transparent information overlay.

The computational resources used by the device may be augmented with additional computation located elsewhere. This remote computation might be used to process information coming from the device (e.g. to perform a computationally intense registration process), it may be used to recall information useful to the function of the device (e.g. to compare this patient with other similar patients to provide “best practice” treatment options), or it may be used to provide information that directs the device (e.g. transferring the indication of a lesion in a CT image to a remote center for biopsy). The use of external computation may be measured and associated with the costs of using the device.

In addition to providing guidance on the needle trajectory, guidance can be provided to indicate the correct depth of penetration. This can be performed by detecting fiducials on the needle, and tracking those fiducials over time. For example, these may be dark rings on the needle itself, which can be counted using the vision system, or they may be a reflective element attached to the end of the needle, and the depth may be computed by subtracting the location of the fiducial in space from the patient surface, and then subtracting that result from the entire length of the needle.

It may also be possible to indicate depth of penetration to the user by projecting a fiducial (e.g. a bright point of light) onto the needle, indicating to what point the needle should be inserted to be at the correct depth.

Additionally, the display of the system may passively indicate the number of fiducial rings that should remain outside the patient at the correct depth for the current system pose, providing the user with a perceptual cue that they can use to determine manually if they are at the correct depth.

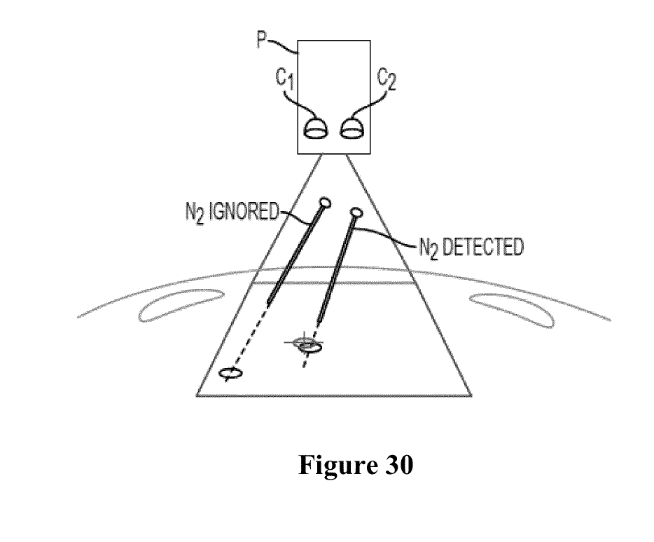

When using the projector for needle guidance, the system may make use of the projected insertion point as “capture range” for possible needle poses, discard candidates outside that range, or detect when computed 3D poses violate the expected targeting behavior, see, e.g., FIG. 30.

For imaging, the PA laser can fire directly and diffusely at the tissue wall, exciting a PA sound wave emanating from there that is received with the mentioned passive US array and can be used for diagnostic purposes. Ideally, using a combination of the mentioned tracking methods, the diagnostic outcome can be linked to a particular location along the GI tract.

Some embodiments of the current invention can allow reconstructing a 2D ultrasound probe's 6-DoF (“degrees of freedom”) trajectory robustly, without the need for an external tracking device. The same mechanism can be e.g. applied to (wireless) capsule endoscopes as well. This can be achieved by cooperative sets of local sensors that incrementally track a probe's location through its sequence of motions. Some aspects of the current invention can be summarized, as follows.

First, an (ultrasound-image-based) speckle decorrelation analysis (SDA) algorithm provides very high-precision 1-DoF translation (distance) information for image patch pairs by decorrelation, and 6-DoF information for the complete ultrasound image when combined with planar 2D-2D registration techniques. Precision of distance estimation is improved by basing the statistics on a larger set of input pairs. (The parallelized approach with a larger input image set can significantly increase speed and reliability.)

Additionally, or alternatively, instead of using a full transmit/receive ultrasound transceiver (e.g. because of space or energy constraints, as in a wireless capsule endoscope), only an ultrasound receiver can be used according to some embodiments of the current invention. The activation energy in this case comes from an embedded laser. Regular laser discharges excite irregularities in the surrounding tissue and generate photoacoustic impulses that can be picked up with the receiver. This can help to track surfaces and subsurface features using ultrasound and thus provide additional information for probe localization.

Second, a component, bracket, or holder housing a set of optical, inertial, and/or capacitive (OIC) sensors represents an independent source of (ultrasound-image-free) motion information. Optical displacement trackers (e.g. from optical mice or cameras) generate local translation data across the scan surface (e.g. skin or intestinal wall), while accelerometers and/or gyroscopes provide absolute orientation and/or rotation motion data. Capacitive sensors can estimate the distance to tissue when the optical sensors loses surface contact or otherwise suffers tracking loss. Their streams of local data are combined over time to reconstruct an n-DoF probe trajectory with n=2 . . . 6, depending on the actual OIC sensor combination and the current pose/motion of the probe.

Third, two or more optical video cameras are attached to the ultrasound probe, possibly in stereo fashion, at vantage points that let them view the surrounding environment, including any or all of the patient skin surface, possible tools and/or needles, possible additional markers, and parts of the operation room environment. This way, they serve to provide calibration, image data registration support, additional tracking input data, additional input data supporting ultrasound elasticity imaging, needle bending detection input, and/or textured 3D environment model data for enhanced visualization.

When used medically, it may be necessary for the camera-projector device to be maintained in a sterile environment. This may be accomplished in a number of ways. The housing may be resistant to sterilizing agents, and perhaps be cleaned by wiping. It may also be placed in a sterile bag cover. In this case, it may be advantageous to create a “window” of solid plastic in the cover that attaches to the cameras and projector. This window may attached mechanically, or magnetically, or by static electric attraction (“static cling”). Another way of maintaining sterility is to produce a sterile (possibly disposable) housing that the projector-camera device mounts into.

One embodiment includes a display system that maintains registration with the probe and which can be used for both visualization and guidance. For example, the probe may have an associated display that the can be detached and which shows relevant pre-operative CT information based on its position in space. It may also overlay targeting information. One example would include a pair of glasses that were registered to the probe and were able to provide “see through” or “heads up” display to the user.

Cameras associated with the augmentation system can be used to perform “quality control” on the overall performance of the system. For example, the trajectory of a needle can be calculated by visual tracking and thence projected into the ultrasound image. If the needle in the image is inconsistent with this projection, it is a cue that there is a system discrepancy. Conversely, if the needle is detected in the ultrasound image, it can be projected back into the video image to confirm that the external pose of the needle is consistent with that tracked image.

According to a further embodiment, the system may simultaneously track the needle in both ultrasound and video images, and to use those computed values to detect needle bending and to either update the likely trajectory of the needle, or to alert the user that they are putting pressure on the needle, or both.

Quality control can also be performed by processing the ultrasound image to determine that it has the expected structure. For example, if the depth setting of the ultrasound machine differs from that expected by the probe, the structure of the image will differ in detectable ways from that expected in this case—for example the wrong amount of “black space” on the image, or wrong annotations on the screen.

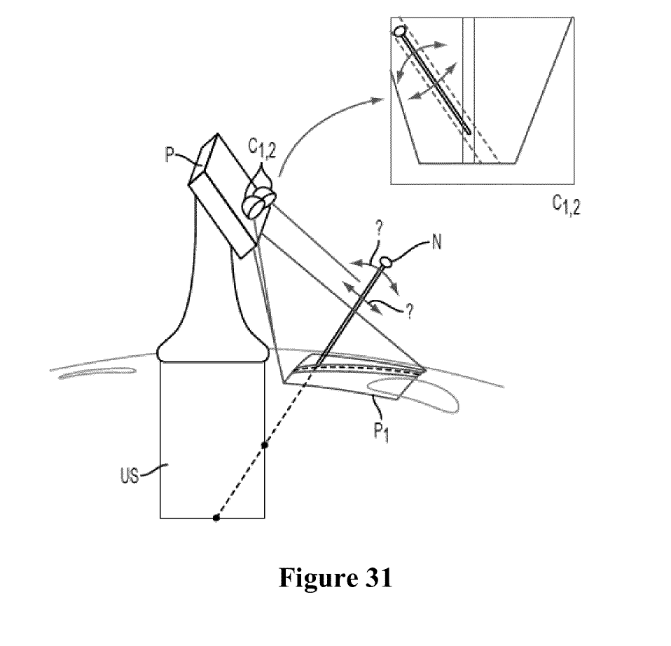

There are a variety of geometries than can be used to provide guidance. In one embodiment, the projection center may lie on or near the plane of the ultrasound system. In this case, the projector can project a single line or shadow that indicates where this plane is. A needle or similar tool placed in the correct plane will become bright or dark, respectively. A video camera outside this plane can view the scene, and this image can be displayed on a screen. Indeed, it may be included with the ultrasound view. In this case, the clinician can view both the external and internal guidance of the needle simultaneously on the same screen. Guidance to achieve a particular angle can be superimposed on the camera image, so that the intersection of the ultrasound plane and the plane formed by the superimposed guidance forms a line that is the desired trajectory of the needle, see, e.g., FIG. 31.

According to another embodiment a camera may be located along the ultrasound plane, and the projector is located off-plane. The geometry is similar, but according to this embodiment, the camera superimposed image is used to define the plane, and a line is projected by the projector to define the needle trajectory.

Further variations include combinations of single or multiple cameras or projectors, where at least one of either is mounted on the mobile device itself as well as mounted statically in the environment, with registration between the mobile and fixed components maintained at all times to make guidance possible. This registration maintenance can be achieved e.g. by detecting and tracking known features present in the environment and/or projected into the common field of interest.

The registration component of the system may take advantage of its ability to “gate” in real time based on patient breathing or heart motion. Indeed, the ability of the probe to monitor surface and subsurface change in real time also means that it could register to “cine” (time-series) MR or CT image, and show that in synchrony with patient motion.

Furthermore, by incorporating additional local sensors (like the OIC sensor bracket) beyond using the ultrasound RF data for the speckle decorrelation analysis (SDA), it is possible to simplify algorithmic complexity and improve robustness by dropping the detection of fully developed speckle (FDS) patches before displacement estimation. While this FDS patch detection is traditionally necessary for SDA, using OIC will provide constraints for the selection of valid patches by limiting the space of possible patches, thus increasing robustness e.g. in combination with RANSAC subset selection algorithms.

Finally, a micro-projection device (laser- or image-projection-based) integrated into the ultrasound probe bracket can provide the operator with an interactive, real-time visualization modality, displaying relevant data like needle intersection points, optimal entry points, and other supporting data directly in the intervention location by projecting these onto the patient skin surface near the probe.

The combination of the camera and projector can be used to construct intuitive and sterile user interfaces on the patient surface, or on any other projectable surface. For example, standard icons and buttons can be projected onto the patient, and a finger or needle can be tracked and used to activate these buttons. This tracking can also be used in non-visual user interfaces, e.g. for gesture tracking without projected visual feedback.

It is another object of the invention to guide the placement of the imaging device on a surface. For example, by making use of the ability of an ultrasound probe or similar imaging device to acquire images from within the body while the video imaging system captures images from outside the body, the probe may be registered in body coordinates. The system may then project guidance as to how to move the probe to visualize a given target. For example, suppose that a tumor is identified in a diagnostic image, or in a previous scan. After registration, the projection system can project an arrow on the patient showing in which direction the probe should move. One of ordinary skill will realize that this method can be used to guide a user to visualize a particular organ based on a prior model of the patient or a patient-specific scan, or could be used to aid in tracking or orienting relative to a given target. For example, it may be desirable to place a gating window (e.g. for Doppler ultrasound) on a particular target or to maintain it therein.

The augmentation system may use multi-band projection with both visible and invisible bands (such as with IR in various ways), simultaneously or time-multiplexed. As noted above, the invention may use multi-projector setups for shadow reduction, intensity enhancement, or passive stereo guidance.

The projection image may be time-multiplexed in synchrony with the camera or cameras to alternatively optimize projection for tracking (maximize needle presence), guidance (overlay clues), surfaces (optimize stereo reconstruction). The projection pattern may also be spatially modulated or multiplexed for different purposes, e.g. projecting a pattern in one area and guidance in other areas.

In order to create a stereo projection, the projection system may make use of mirrors for making one projector two (or more) by using “arms” etc. to split the image or to accomplish omnidirectional projection, see, e.g., FIG. 32.

The projection system may make use of polarization for 3D guidance or use dual-arm or dual-device projection with polarized light and (passive) glasses for 3D in-situ ultrasound guidance display. The projection may project onto a screen, including a fog screen, switchable film, and UV-fluorescent glass, as almost-in-situ projection surfaces

The projection system may make use of the geometry computed by the stereo system to correct for the curvature of the body when projecting information onto it.

The projection system may include outward-looking cameras to track the user to help correct visualization from geometric distortion or probe motion. This may also be used to solve the parallax problem when projecting in 3D.

The projection system may project a fixed pattern upwards onto the environment to support tracking with stereo cameras (limited degrees of freedom, depending on environment structure). The projection system may project a fixed pattern upwards onto the environment to support tracking with stereo cameras. The system may make use of 3D information that is computed from the projected pattern, it may make use of image appearance information that comes from objects in the world, or it may use both appearance and depth information. It may be useful to synchronize the projection in such a way that images with the pattern and without are obtained. Methods for performing 3D reference positioning using depth and intensity information are well known in the art.

The projector may make use of light-activated dyes that have been “printed on patient” or may contain an auxiliary controlled laser for this purpose.

Rather than relying on the patient surface as a projection surface, the projector might instead project onto other rigid or deformable objects in the workspace. For example, the camera may reconstruct a sheet of paper in space, and the projector could project the CT data of a preoperative scan onto the paper. As the paper is deformed the CT data would be altered to reflect the data that it would “slice through” if it were inside the body. This would allow the visualization of curved surfaces or curvilinear structures.

It is often the case that a patient is imaged multiple times, for example to provide guidance for radiative cancer therapy. In this case, the images around the target could be recorded, and, upon subsequent imaging, these images would be used to provide guidance on how to move the probe toward a desired target, and an indication when the previous imaging position is reached.

In order to improve the usability of these methods, the system may have an electronic or printable signature that records the essential targeting information in an easy-to-use way. This information may be loaded or scanned visually by the device itself when the patient is re-imaged.

An interesting use of the above method of probe and needle guidance is to make ultrasound treatment accessible for non-experts. This may include providing training for those learning about diagnostic or interventional ultrasound, or to make it possible for the general population to make use of ultrasound-based treatments for illness. These methods could also monitor the use of an imaging probe and/or needles etc. and indicate when the user is poorly trained.

An example of the application of the above would be to have an ultrasound system installed at a pharmacy, and to perform automated carotid artery examination by an unskilled user.

There are many other applications for these ideas that extend beyond ultrasound and medicine. For example, nondestructive inspection of a plane wing may use ultrasound or x-ray, but in either case requires exact guidance to the inspection location (e.g. a wing attachment) in question. The methods described above can provide this guidance. In a more common setting, the system could provide guidance for e.g. throwing darts, hitting a pool ball, or a similar game.

The embodiments illustrated and discussed in this specification are intended only to teach those skilled in the art the best way known to the inventors to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

### Example 1

### Ultrasound-Guided Liver Ablation Therapy

Recent evidence suggests thermal ablation in some cases can achieve results comparable to that of resection. Specifically, a recent randomized clinical trial comparing resection to RFA for small HCC found equivalent long-term outcomes with lower morbidity in the ablation arm [Chen-2006] Importantly, most studies suggest that efficacy of RFA is highly dependent on the experience and diligence of the treating physician, often associated with a steep learning curve [Poon-2004]. Moreover, the apparent efficacy of open operative RFA over a percutaneous approach reported by some studies suggest that difficulty with targeting and imaging may be contributing factors [Mulier-2005]. Studies of the failure patterns following RFA similarly suggest that limitations in real-time imaging, targeting, monitoring of ablative therapy are likely contributing to increased risk of local recurrence [Mulier-2005].

One of the most useful features of ablative approaches such as RFA is that it can be applied using minimally invasive techniques. Length of hospital stay, costs, and morbidity may be reduced using this technique [Berber-2008]. These benefits add to the appeal of widening the application of local therapy for liver tumors to other tumor types, perhaps in combination with more effective systemic therapies for minimal residual disease Improvements in the control, size, and speed of tumor destruction with RFA will begin to allow us to reconsider treatment options for such patients with liver tumors as well. However, clinical outcomes data are clear—complete tumor destruction with adequate margins is imperative in order to achieve durable local control and survival benefit, and this should be the goal of any local therapy. Partial, incomplete, or palliative local therapy is rarely indicated. One study even suggested that incomplete destruction with residual disease may in fact be detrimental, stimulating tumor growth of locally residual tumor cells [Koichi-2008]. This concept is often underappreciated when considering tumor ablation, leading to lack of recognition by some of the importance of precise and complete tumor destruction. Improved targeting, monitoring, and documentation of adequate ablation are critical to achieve this goal. Goldberg et al, in the most cited work on this subject [Goldberg-2000], describes an ablative therapy framework in which the key areas in advancing this technology include improving (1) image guidance, (2) intra-operative monitoring, as well as (3) ablation technology itself.

In spite of promising results of ablative therapies, significant technical barriers exist with regard to its efficacy, safety, and applicability to many patients. Specifically, these limitations include: (1) localization/targeting of the tumor and (2) monitoring of the ablation zone.

Targeting Limitations: One common feature of current ablative methodology is the necessity for precise placement of the end-effector tip in specific locations, typically within the volumetric center of the tumor, in order to achieve adequate destruction. The tumor and zone of surrounding normal parenchyma can then be ablated. Tumors are identified by preoperative imaging, primarily CT and MR, and then operatively (or laparoscopically) localized by intra-operative ultrasonography (IOUS). When performed percutaneously, trans-abdominal ultrasonography is most commonly used. Current methodology requires visual comparison of preoperative diagnostic imaging with real-time procedural imaging, often requiring subjective comparison of cross-sectional imaging to IOUS. Then, manual free-hand IOUS is employed in conjunction with free-hand positioning of the tissue ablator under ultrasound guidance. Target motion upon insertion of the ablation probe makes it difficult to localize appropriate placement of the therapy device with simultaneous target imaging. The major limitation of ablative approaches is the lack of accuracy in probe localization within the center of the tumor. This is particularly important, as histological margins cannot be assessed after ablations as opposed to hepatic resection approaches [Koniaris-2000] [Scott-2001]. In addition, manual guidance often requires multiple passes and repositioning of the ablator tip, further increasing the risk of bleeding and tumor dissemination. In situations when the desired target zone is larger than the single ablation size (e.g. 5-cm tumor and 4-cm ablation device), multiple overlapping spheres are required in order to achieve complete tumor destruction. In such cases, the capacity to accurately plan multiple manual ablations is significantly impaired by the complex 3D geometrically complex planning required as well as image distortion artifacts from the first ablation, further reducing the targeting confidence and potential efficacy of the therapy. IOUS often provides excellent visualization of tumors and guidance for probe placement, but its 2D-nature and dependence on the sonographer's skills limit its effectiveness [Wood-2000].

Improved real-time guidance for planning, delivery and monitoring of the ablative therapy would provide the missing tool needed to enable accurate and effective application of this promising therapy. Recent studies are beginning to identify reasons for diminished efficacy of ablative approaches, including size, location, operator experience, and technical approach [Mulier-2005] [van Duijnhoven-2006]. These studies suggest that device targeting and ablation monitoring are likely the key reasons for local failure. Also, due to gas bubbles, bleeding, or edema, IOUS images provide limited visualization of tumor margins or even the applicator electrode position during RFA [Hinshaw-2007].

The impact of radiological complete response on tumor targeting is an important emerging problem in liver directed therapy. Specifically, this problem relates to the inability to identify the target tumor at the time of therapy. Effective combination systemic chemotherapeutic regimens are being used with increasing frequency prior to liver-directed therapy to treat potential micro-metastatic disease as a neo-adjuvant approach, particularly for colorectal metastases [Gruenberger-2008]. This allows the opportunity to use the liver tumor as a gauge to determine chemo-responsiveness as an aid to planning subsequent post-procedural chemotherapy. However, in such an approach, the target lesion often cannot be identified during the subsequent resection or ablation. We know that even when the index liver lesion is no longer visible, microscopic tumors are still present in more than 80% of cases [Benoist-2006]. Any potentially curative approach, therefore, still requires complete resection or local destruction of all original sites of disease. In such cases, the interventionalist can face the situation of contemplating a “blind” ablation in region of the liver in which no imagable tumor can be detected. Therefore, without an ability to identify original sites of disease, preoperative systemic therapies may actually hinder the ability to achieve curative local targeting, paradoxically potentially worsening long-term survival. As proposed in this project, integrating a strategy for registration of the pre-chemotherapy cross-sectional imaging (CT) with the procedure-based imaging (IOUS) would provide invaluable information for ablation guidance.

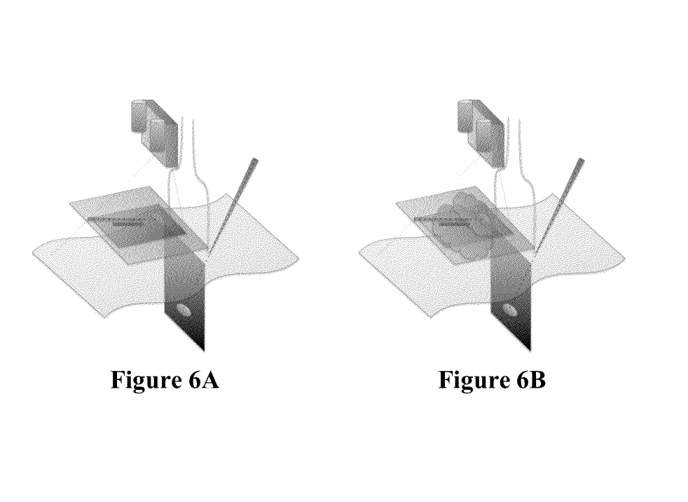

Our system embodiments described both in FIG. 1 and FIG. 2 can be utilized in the above mentioned application. With structured light attached to the ultrasound probe, patient surface can be captured and digitized in real-time. Then, the doctor will select an area of interest to scan where he/she can observe a lesion either directly from the ultrasound images or indirectly from the fused pre-operative data. The fusion is performed by integrating both surface data from structured light and few ultrasound images and can be updated in real-time without manual input from the user. Once the lesion is identified in the US probe space, the doctor can introduce the ablation probe, where the SLS system can easily segment/track and localize the tool before inserting to the patient (FIG. 9). The projector can be used to overlay real-time guidance information to help orient the tool and provide a feedback about the needed insertion depth.

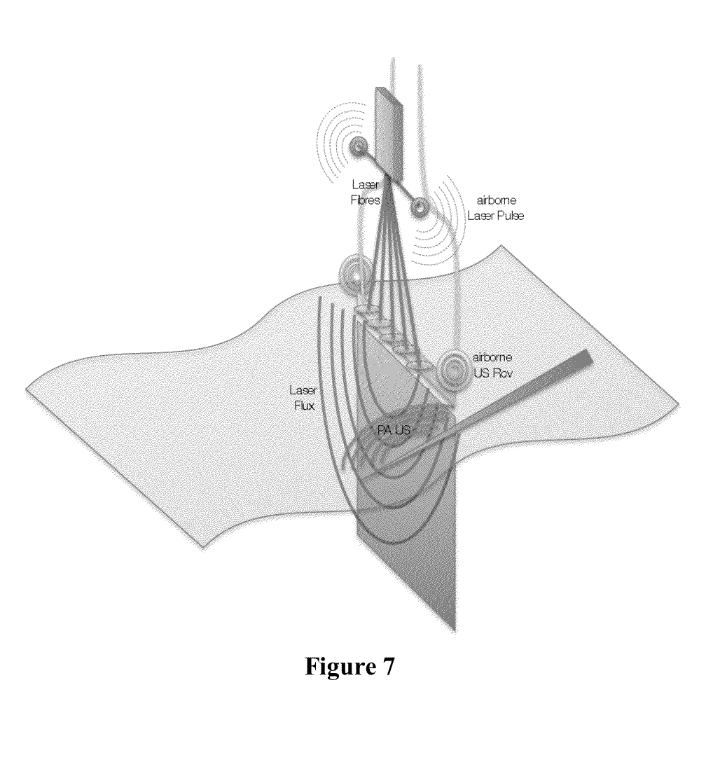

Abovementioned is the embodiment described in FIG. 1. However, our invention includes many alternates for example: 1) Time-of-flight camera can replace the SLS configuration to provide the surface data [Billings-2011] (FIG. 10). In this embodiment, the ToF camera is not attached to the ultrasound probe, and an external tracker is used to track both components. Projector can still be attached to the ultrasound probe. 2) Another embodiment consists of SLS or ToF camera to provide surface information and a projector attached to the ultrasound probe. The camera configuration, i.e. SLS should be able to extract surface data, track intervention tool, and probe surface, hence can locate the needle to the US image coordinate. This embodiment requires offline calibration to estimate the transformation between the probe surface shape and the actual location of the ultrasound image. A projector still can be used to overlay needle location and visualize guidance information. 3) Furthermore, embodiment can only consist of projectors and local sensors. FIG. 7 describes a system composed of pulsed laser projector to track an interventional tool in air and in tissue using photoacoustic (PA) phenomenon [Boctor-2010]. Interventional tools can convert pulsed light energy into an acoustic wave that can be picked up by multiple acoustic sensors placed on the probe surface, which we then can apply known triangulation algorithms to locate the needle. It is important to note that one can apply laser light directly to the needle, i.e. attach fiber optic configuration to a needle end; the needle can also conduct the generated acoustic wave (i.e. acting like a wave-guide) and fraction of this acoustic wave can propagate from the needle shaft and tip and the PA signals, i.e. acoustic signals generated, can be picked up by both sensors attached to the surface as well as the ultrasound array elements. In addition to the laser light projecting directly to the needle, we can extend few fibers to deposit light energy underneath the probe, hence can track the needle inside the tissue (FIG. 7).

One possible embodiment is to integrate both an ultrasound probe with an endoscopic camera held on one endoscopic channel and having the projector component connected in a separate channel. This projector can enable structured light, and the endoscopic camera performs surface estimation to help performing hybrid surface/ultrasound registration with a pre-operative modality. Possibly, the projector can be a pulsed laser projector that can enable PA effects and the ultrasound probe attached to the camera can generate PA images for region of interest.

### REFERENCES

[Benoist-2006] Benoist S, Brouquet A, Penna C, Julié C, El Hajjam M, Chagnon S, Mitry E, Rougier P, Nordlinger B, “Complete response of colorectal liver metastases after chemotherapy: does it mean cure?” J Clin Oncol. 2006 Aug. 20; 24(24):3939-45.
[Berber-2008] Berber E, Tsinberg M, Tellioglu G, Simpfendorfer C H, Siperstein A E. Resection versus laparoscopic radiofrequency thermal ablation of solitary colorectal liver metastasis. J Gastrointest Surg. 2008 November; 12(11): 1967-72.
[Billings-2011] Billings S, Kapoor A, Wood B J, Boctor E M, “A hybrid surface/image based approach to facilitate ultrasound/CT registration,” accepted SPIE Medical Imaging 2011.
[Boctor-2010] E. Boctor, S. Verma et al. “Prostate brachytherapy seed localization using combined photoacoustic and ultrasound imaging,” SPIE Medical Imaging 2010.
[Chen-2006] Chen M S, Li J Q, Zheng Y, Guo R P, Liang H H, Zhang Y Q, Lin X J, Lau W Y. A prospective randomized trial comparing percutaneous local ablative therapy and partial hepatectomy for small hepatocellular carcinoma. Ann Surg. 2006 March; 243(3):321-8.
[Goldberg-2000] Goldberg S N, Gazelle G S, Mueller P R. Thermal ablation therapy for focal malignancy: a unified approach to underlying principles, techniques, and diagnostic imaging guidance. AJR Am J. Roentgenol. 2000 February; 174(2):323-31.
[Gruenberger-2008] Gruenberger B, Scheithauer W, Punzengruber R, Zielinski C, Tamandl D, Gruenberger T Importance of response to neoadjuvant chemotherapy in potentially curable colorectal cancer liver metastases. BMC Cancer. 2008 Apr. 25; 8:120.
[Hinshaw-2007] Hinshaw J L, et. al., Multiple-Electrode Radiofrequency Ablation of Symptomatic Hepatic Cavernous Hemangioma, Am. J. Roentgenol., Vol. 189, Issue 3, W-149, Sep. 1, 2007.
[Koichi-2008] Koichi O, Nobuyuki M, Masaru O et al., “Insufficient radiofrequency ablation therapy may induce further malignant transformation of hepatocellular carcinoma,” Journal of Hepatology International, Volume 2, Number 1, March 2008, pp 116-123.
[Koniaris-2000] Koniaris L G, Chan D Y, Magee C, Solomon S B, Anderson J H, Smith D O, DeWeese T, Kavoussi L R, Choti M A, “Focal hepatic ablation using interstitial photon radiation energy,” J Am Coll Surg. 2000 August; 191(2):164-74.
[Mulier-2005] Mulier S, Ni Y, Jamart J, Ruers T, Marchal G, Michel L. Local recurrence after hepatic radiofrequency coagulation: multivariate meta-analysis and review of contributing factors. Ann Surg. 2005 August; 242(2):158-71.
[Poon-2004] Poon R T, Ng K K, Lam C M, Ai V, Yuen J, Fan S T, Wong J. Learning curve for radiofrequency ablation of liver tumors: prospective analysis of initial 100 patients in a tertiary institution. Ann Surg. 2004 April; 239(4):441-9.
[Scott-2001] Scott D J, Young W N, Watumull L M, Lindberg G, Fleming J B, Huth J F, Rege R V, Jeyarajah D R, Jones D B, “Accuracy and effectiveness of laparoscopic vs open hepatic radiofrequency ablation,” Surg Endosc. 2001 February; 15(2):135-40.
[van Duijnhoven-2006] van Duijnhoven F H, Jansen M C, Junggeburt J M, van Hillegersberg R, Rijken A M, van Coevorden F, van der Sijp J R, van Gulik T M, Slooter G D, Klaase J M, Putter H, Tollenaar R A, “Factors influencing the local failure rate of radiofrequency ablation of colorectal liver metastases,” Ann Surg Oncol. 2006 May; 13(5):651-8. Epub 2006 Mar. 17.
[Wood-2000] Wood T F, Rose D M, Chung M, Allegra D P, Foshag L J, Bilchik A J, “Radiofrequency ablation of 231 unresectable hepatic tumors: indications, limitations, and complications,” Ann Surg Oncol. 2000 September; 7(8):593-600.

### Example 2

### Monitoring Neo-Adjuvant Chemotherapy Using Advanced Ultrasound Imaging

Out of more than two hundred thousand women diagnosed with breast cancer every year, about 10% will present with locally advanced disease [Valero-1996]. Primary chemotherapy (a.k.a. Neo-adjuvant chemotherapy, NAC) is quickly replacing adjuvant (post-operative) chemotherapy as the standard in the management of these patients. In addition, NAC is often administered to women with operable stage II or III breast cancer [Kaufmann-2006]. The benefit of NAC is two fold. First, NAC has the ability to increase the rate of breast conserving therapy. Studies have shown that more than fifty percent of women, who would otherwise be candidates for mastectomy only, become eligible for breast conserving therapy because of NAC induced tumor shrinkage [Hortabagyi-1988, Bonadonna-1998]. Second, NAC allows in vivo chemo-sensitivity assessment. The ability to detect early drug resistance will prompt change from the ineffective to an effective regimen. Consequently, physicians may decrease toxicity and perhaps improve outcome. The metric most commonly used to determine in-vivo efficacy is the change in the tumor sized during NAC.

Unfortunately, the clinical tools used to measure tumor size during NAC, such as physical exam, mammography, and B-mode ultrasound, have been shown to be less than ideal. Researchers have shown that post-NAC tumor size estimates by physical exam, ultrasound and mammography, when compared to pathologic measurements, have correlation coefficients of 0.42, 0.42, and 0.41 respectively [Chagpar-2006]. MRI and PET appear to be more predictive of response to NAC however these modalities are expensive, inconvenient and, with respect to PET, impractical for serial use due to excessive radiation exposure [Smith-2000, Rosen-2003, Partridge-2002]. What is needed is an inexpensive, convenient and safe technique capable of accurately measuring tumor response repeatedly during NAC.

Ultrasound is a safe modality which easily lends itself to serial use. However, the most common system currently in medical use, B-Mode ultrasound, does not appear to be sensitive enough to determine subtle changes in tumor size. Accordingly, USEI has emerged as a potentially useful augmentation to conventional ultrasound imaging. USEI has been made possible by two discoveries: (1) different tissues may have significant differences in their mechanical properties and (2) the information encoded in the coherent scattering (a.k.a. speckle) may be sufficient to calculate these differences following a mechanical stimulus [Ophir-1991]. An array of parameters, such as velocity of vibration, displacement, strain, velocity of wave propagation and elastic modulus, have been successfully estimated [Konofagou-2004, Greenleaf-2003], which then made it possible to delineate stiffer tissue masses, such as tumors [Hall-2002, Lyshchik-2005, Purohit-2003], ablated lesions [Varghese-2004, Boctor-2005]. Breast cancer detection is the first [Garra-1997] and most promising [Hall-2003] application of USEI.

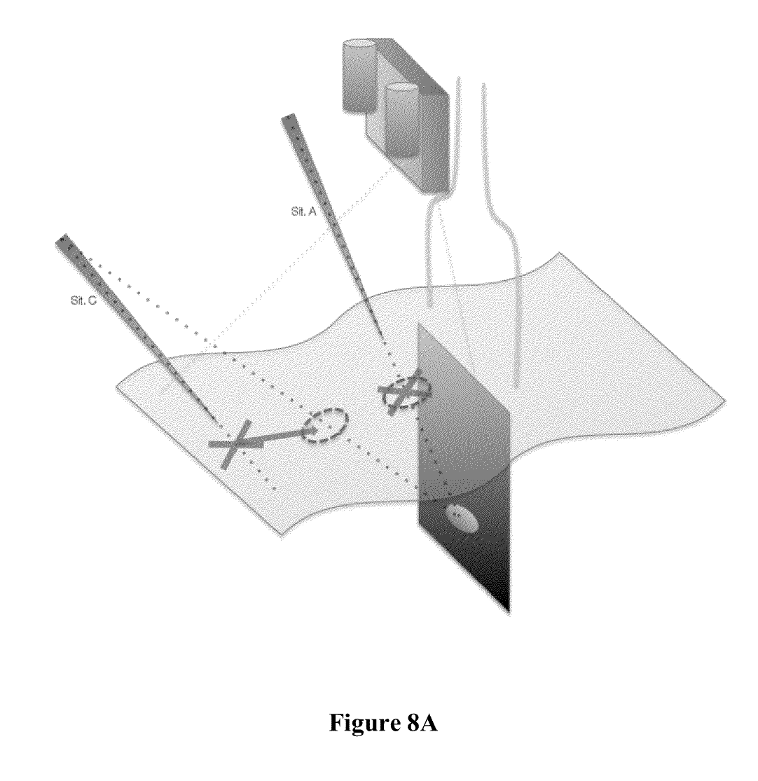

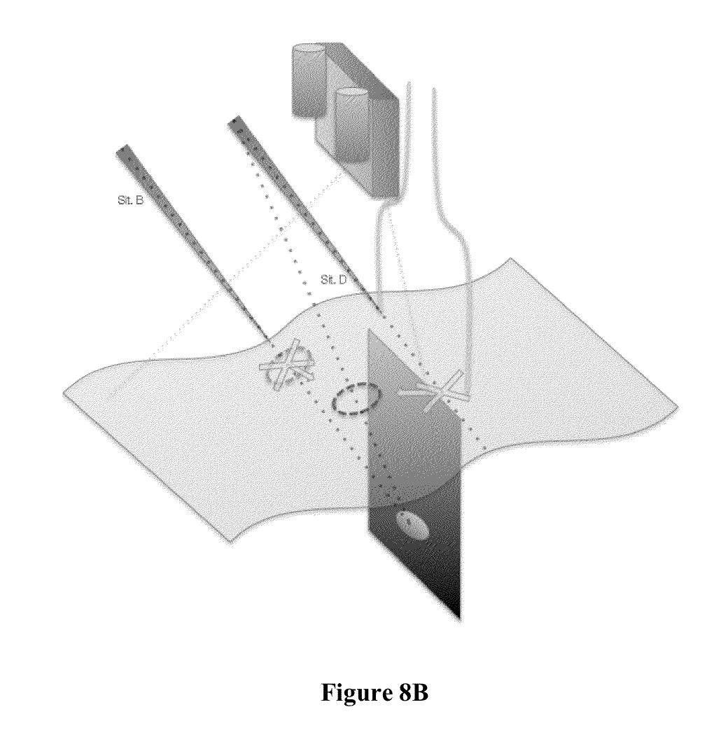

An embodiment for this application is to use an ultrasound probe and an SLS configuration attached to the external passive arm. We can track both the SLS and the ultrasound probe using external tracking device, or simply use the SLS configuration to track the probe with respect to SLS's own reference frame. On day one, we place the probe one the region of interest and the SLS configuration captures the breast surface information, the ultrasound probe surface and provides a substantial input for the following task: 1) The US probe can be tracked and hence 3D US volume can be reconstructed from 2D images (the US probe is a 2D probe); or the resulting small volumes from a 3D probe can be stitched together and form a panoramic volume, 2). The US probe can be tracked during elastography scan. This tracking information can be integrated in the EI algorithm to enhance the quality [Foroughi-2010] (FIG. 11), and 3) Registration between ultrasound probe's location on the first treatment session and subsequent sessions can be easily recovered using the SLS surface information (as shown in FIG. 12) for both the US probe and the breast.

### REFERENCES

[Boctor-2005] Boctor E M, DeOliviera M, Awad M., Taylor R H, Fichtinger G, Choti M A, Robot-assisted 3D strain imaging for monitoring thermal ablation of liver, Annual congress of the Society of American Gastrointestinal Endoscopic Surgeons, pp 240-241, 2005.
[Bonadonna-1998] Bonadonna G, Valagussa P, Brambilla C, Ferrari L, Moliterni A, Terenziani M, Zambetti M, “Primary chemotherapy in operable breast cancer: eight-year experience at the Milan Cancer Institute,” SOJ Clin Oncol 1998 January; 16(1):93-100.
[Chagpar-2006] Chagpar A, et al., “Accuracy of Physical Examination, Ultrasonography and Mammogrpahy in Predicting Residual Pathologic Tumor size in patients treated with neoadjuvant chemotherapy” Annals of surgery Vol. 243, Number 2, February 2006.
[Greenleaf-2003] Greenleaf J F, Fatemi M, Insana M. Selected methods for imaging elastic properties of biological tissues. Annu Rev Biomed Eng. 2003; 5:57-78.
[Hall-2002] Hall T J, Yanning Zhu, Spalding C S “In vivo real-time freehand palpation imaging Ultrasound Med Biol. 2003 March; 29(3):427-35.
[Konofagou-2004] Konofagou E E. Quovadis elasticity imaging? Ultrasonics. 2004 April; 42(1-9):331-6.
[Lyshchik-2005] Lyshchik A, Higashi T, Asato R, Tanaka S, Ito J, Mai J J, Pellot-Barakat C, Insana M F, Brill A B, Saga T, Hiraoka M, Togashi K. Thyroid gland tumor diagnosis at US elastography. Radiology. 2005 October; 237(1):202-11.
[Ophir-1991] Ophir J, Céspedes E I, Ponnekanti H, Yazdi Y, Li X: Elastography: a quantitative method for imaging the elasticity of biological tissues. Ultrasonic Imag., 13:111-134, 1991.
[Partridge-2002] Partridge S C, Gibbs J E, Lu Y, Esserman L J, Sudilovsky D, Hylton N M, “Accuracy of MR imaging for revealing residual breast cancer in patients who have undergone neoadjuvant chemotherapy,” AJR Am J. Roentgenol. 2002 November; 179(5):1193-9.
[Purohit-2003] Purohit R S, Shinohara K, Meng M V, Carroll P R. Imaging clinically localized prostate cancer. Urol Clin North Am. 2003 May; 30(2):279-93.
[Rosen-2003] Rosen E L, Blackwell K L, Baker J A, Soo M S, Bentley R C, Yu D, Samulski T V, Dewhirst M W, “Accuracy of MRI in the detection of residual breast cancer after neoadjuvant chemotherapy,” AJR Am J. Roentgenol. 2003 November; 181(5):1275-82.
[Smith-2000] Smith I C, Welch A E, Hutcheon A W, Miller I D, Payne S, Chilcott F, Waikar S, Whitaker T, Ah-See A K, Eremin O, Heys S D, Gilbert F J, Sharp P F, “Positron emission tomography using [(18)F]-fluorodeoxy-D-glucose to predict the pathologic response of breast cancer to primary chemotherapy,” J Clin Oncol. 2000 April; 18(8):1676-88.
[Valero-1996] Valero V, Buzdar A U, Hortobagyi G N, “Locally Advanced Breast Cancer,” Oncologist. 1996; 1(1 & 2):8-17.
[Varghese-2004] Varghese T, Shi H. Elastographic imaging of thermal lesions in liver in-vivo using diaphragmatic stimuli. Ultrason Imaging. 2004 January; 26(1):18-28.
[Foroughi-2010] P. Foroughi, H. Rivaz, I. N. Fleming, G. D. Hager, and E. Boctor, “Tracked Ultrasound Elastography (TrUE),” in Medical Image Computing and Computer Integrated surgery, 2010.

### Example 3

### Ultrasound Imaging Guidance for Laparoscopic Partial Nephrectomy

Kidney cancer is the most lethal of all genitourinary tumors, resulting in greater than 13,000 deaths in 2008 out of 55,000 new cases diagnosed [61]. Further, the rate at which kidney cancer is diagnosed is increasing [1,2,62]. “Small” localized tumors currently represent approximately 66% of new diagnoses of renal cell carcinoma [63].

Surgery remains the current gold standard for treatment of localized kidney tumors, although alternative therapeutic approaches including active surveillance and emerging ablative technologies [5] exist. Five year cancer-specific survival for small renal tumors treated surgically is greater than 95% [3,4]. Surgical treatments include simple nephrectomy (removal of the kidney), radical nephrectomy (removal of the kidney, adrenal gland, and some surrounding tissue) and partial nephrectomy (removal of the tumor and a small margin of surrounding tissue, but leaving the rest of the kidney intact). More recently, a laparoscopic option for partial nephrectomy (LPN) has been developed with apparently equivalent cancer control results compared to the open approach [9,10]. The benefits of the laparoscopic approach are improved cosmesis, decreased pain, and improved convalescence relative to the open approach.

Although a total nephrectomy will remove the tumor, it can have serious consequences for patients whose other kidney is damaged or missing or who are otherwise at risk of developing severely compromised kidney function. This is significant given the prevalence of risk factors for chronic renal failure such as diabetes and hypertension in the general population [7,8]. Partial nephrectomy has been shown to be oncologically equivalent to total nephrectomy removal for treatment of renal tumors less than 4 cm in size (e.g., [3,6]). Further, data suggest that patients undergoing partial nephrectomy for treatment of their small renal tumor enjoy a survival benefit compared to those undergoing radical nephrectomy [12-14]. A recent study utilizing the Surveillance, Epidemiology and End Results cancer registry identified 2,991 patients older than 66 years who were treated with either radical or partial nephrectomy for renal tumors <4 cm [12]. Radical nephrectomy was associated with an increased risk of overall mortality (HR 1.38, p<0.01) and a 1.4 times greater number of cardiovascular events after surgery compared to partial nephrectomy.

Despite the advantages in outcomes, partial nephrectomies are performed in only 7.5% of cases [11]. One key reason for this disparity is the technical difficulty of the procedure. The surgeon must work very quickly to complete the resection, perform the necessary anastamoses, and restore circulation before the kidney is damaged. Further, the surgeon must know where to cut to ensure cancer-free resection margins while still preserving as much good kidney tissue as possible. In performing the resection, the surgeon must rely on memory and visual judgment to relate preoperative CT and other information to the physical reality of the patient's kidney. These difficulties are greatly magnified when the procedure is performed laparoscopically, due to the reduced dexterity associated with the instruments and reduced visualization from the laparoscope.

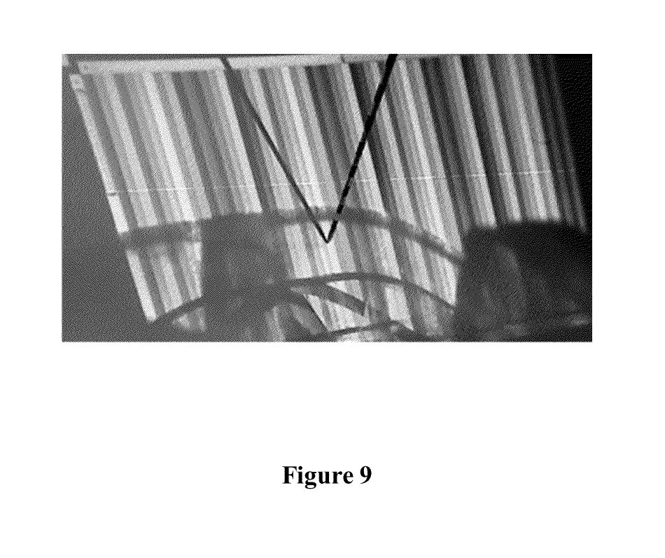

We devised two embodiments to overcome this technically challenging intervention. FIG. 13 shows the first system where an SLS component is held on a laparoscopic arm, a laparoscopic ultrasound probe and an external tracking device to track both the US probe and the SLS [Stolka-2010]. However, we don't need to rely on an external tracking device since we have access to an SLS configuration. SLS can scan kidney surface and probe surface and track both kidney and the US probe. Furthermore, our invention is concerned with Hybrid surface/ultrasound registration. In this embodiment the SLS will scan the kidney surface and together with few ultrasound images a reliable registration with pre-operative data can be performed and augmented visualization, similar to the one shown in FIG. 13, can be visualized using the attached projector.

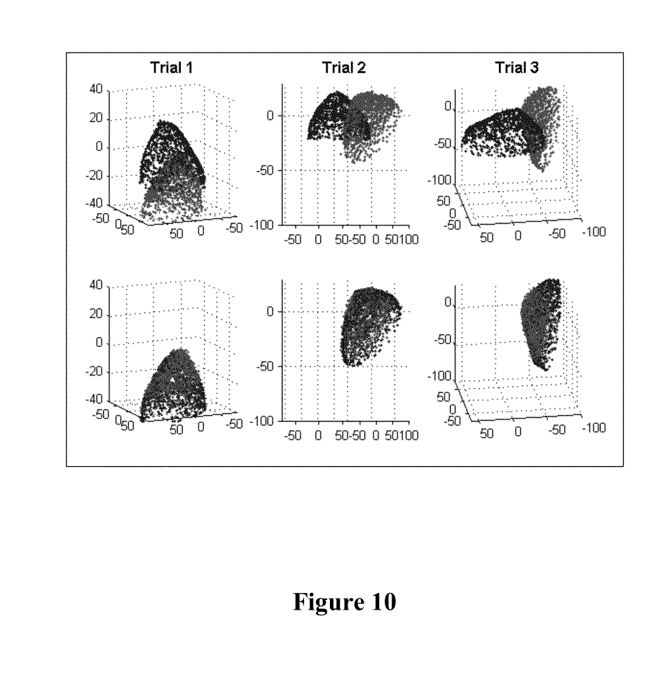

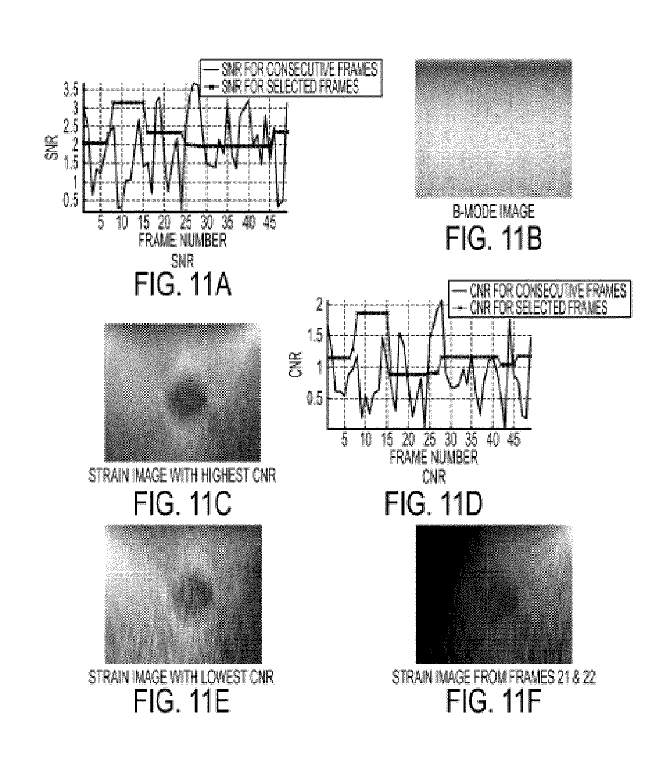

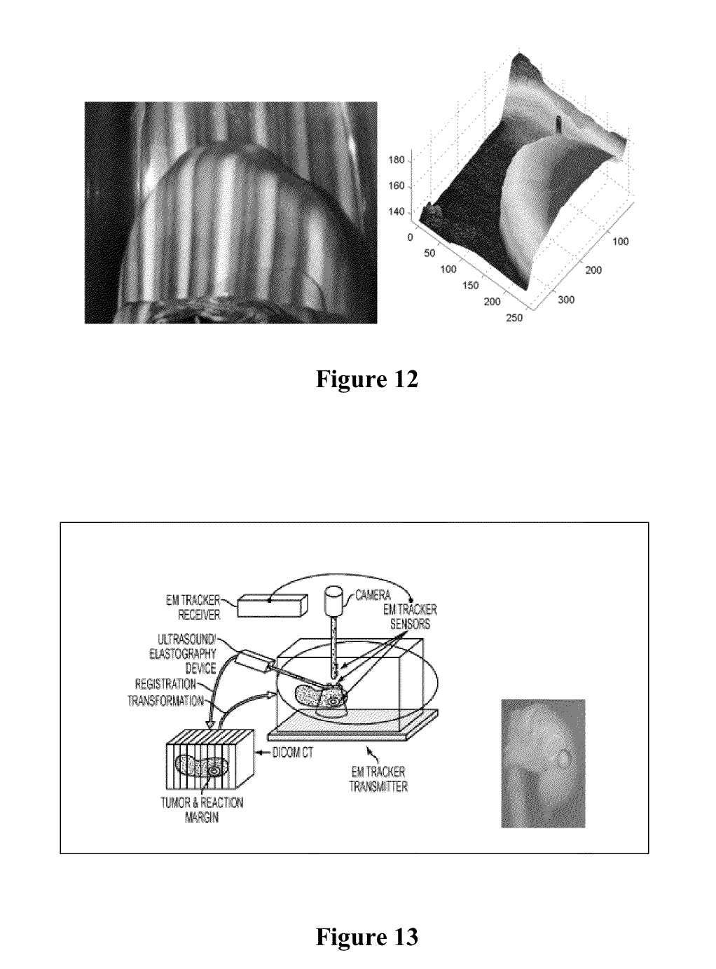

The second embodiment is shown in FIG. 14 where an ultrasound probe is located outside the patient and facing directly towards the superficial side of the kidney. Internally a laparoscopic tool holds an SLS configuration. The SLS system provides kidney surface information in real-time and the 3DUS also images the same surface (tissue-air interface). By applying surface-to-surface registration ultrasound volume can be easily registered to the SLS reference frame. In a different embodiment, registration can be also performed using photoacoustic effect (FIG. 15). Typically, the project in the SLS configuration can be a pulsed laser projector with a fixed pattern. Photoacoustic signals will be generated at specified points, which forms a known calibrated pattern. The ultrasound imager can detect these points PA signals. Then a straightforward point-to-point registration can be performed to establish real-time registration between the camera/projector-space and the ultrasound space.

C-Arm-Guided Interventional Application

Projection data truncation problem is a common issue with reconstructed CT and C-arm images. This problem appears clearly near the image boundaries. Truncation is a result of the incomplete data set obtained from the CT/C-arm modality. An algorithm to overcome this truncation error has been developed [Xu-2010]. In addition to the projection data, this algorithm requires the patient contour in 3D space with respect to the X-Ray detector. This contour is used to generate the trust region required to guide the reconstruction method. A simulation study on a digital phantom was done [Xu-2010] to reveal the enhancement achieved by the new method. However, a practical way to get the trust region has to be developed. FIG. 3 and FIG. 4 present novel practical embodiments to track and to obtain the patient contour information and consequentially the trust region at each view angle of the scan. The trust region is used to guide the reconstruction method [Ismail-2011].

It is known that X-ray is not ideal modality for soft-tissue imaging. Recent C-arm interventional systems are equipped with flat-panel detectors and can perform cone-beam reconstruction. The reconstruction volume can be used to register intraoperative X-ray data to pre-operative MRI. Typically, couple of hundreds X-ray shots need to be taken in order to perform the reconstruction task. Our novel embodiments are capable of performing surface-to-surface registration by utilizing real-time and intraoperative surfaces from SLS or ToF or similar surface scanner sensors. Hence, reducing X-ray dosage is achieved. Nevertheless, if there is need to fine tune the registration task, in this case few X-rays images can be integrated in the overall framework.

It is obvious that similar to US navigation examples and methods described before, the SLS component configured and calibrated to a C-arm can also track interventional tools and the projector attached can provide real-time visualization.

Furthermore, ultrasound probe can be easily introduced to the C-arm scene without adding or changing the current setup. The SLS configuration is capable of tracking the US probe. It is important to note that in many pediatric interventional applications, there is need to integrate ultrasound imager to the C-arm suite. In these scenarios, the SLS configuration can be either attached to the C-arm, to the ultrasound probe, or separately attached to an arm. This ultrasound/C-arm system can consist of more than one SLS configuration, or combination of these sensors. For example, the camera or multiple cameras can be fixed to the C-arm where the projector can be attached to the US probe.

Finally, our novel embodiment can provide quality control to the C-arm calibration. C-arm is a moving equipment and can't be considered a rigid-body, i.e. there is a small rocking/vibrating motion that need to be measured/calibrated at the manufacture site and these numbers are used to compensate during reconstruction. If a faulty condition happened that alter this calibration, the company needs to be informed to re-calibrate the system. These faulty conditions are hard to detect and repeated QC calibration is also unfeasible and expensive. Our accurate surface tracker should be able to determine the motion of the C-arm and continuously, in the background, compare to the manufacture calibration. Once a faulty condition happens, our system should be able to discover and possible correct it.

### REFERENCES

[Jemal-2007] Jemal A, Siegel R, Ward E, Murray T, Xu J, Thun M J. Cancer statistics, 2007. CA Cancer J Clin 2007 January-February; 57(1):43-66.
2. [Volpe-2004] Volpe A, Panzarella T, Rendon R A, Haider M A, Kondylis F I, Jewett M A. The natural history of incidentally detected small renal masses. Cancer 2004 Feb. 15; 100(4):738-45
3. [Fergany-2000] Fergany A F, Hafez K S, Novick A C. Long-term results of nephron sparing surgery for localized renal cell carcinoma: 10-year followup. J Urol 2000 February; 163(2):442-5.
4. [Hafez-1999] Hafez K S, Fergany A F, Novick A C. Nephron sparing surgery for localized renal cell carcinoma: impact of tumor size on patient survival, tumor recurrence and TNM staging. J Urol 1999 December; 162(6):1930-3.
5. [Kunkle-2008] Kunkle D A, Egleston B L, Uzzo R G. Excise, ablate or observe: the small renal mass dilemma—a meta-analysis and review. J Urol 2008 April; 179(4):1227-33; discussion 33-4.
6. [Leibovich-2004] Leibovich B C, Blute M L, Cheville J C, Lohse C M, Weaver A L, Zincke H. Nephron sparing surgery for appropriately selected renal cell carcinoma between 4 and 7 cm results in outcome similar to radical nephrectomy. J Urol 2004 March; 171(3):1066-70.
7. [Coresh-2007] Coresh J, Selvin E, Stevens L A, Manzi J, Kusek J W, Eggers P, et al. Prevalence of chronic kidney disease in the United States. JAMA 2007 Nov. 7; 298(17):2038-47.
8. [Bijol-2006] Bijol V, Mendez G P, Hurwitz S, Rennke H G, Nose V. Evaluation of the normeoplastic pathology in tumor nephrectomy specimens: predicting the risk of progressive renal failure. Am J Surg Pathol 2006 May; 30(5):575-84.
9. [Allaf-2004] Allaf M E, Bhayani S B, Rogers C, Varkarakis I, Link R E, Inagaki T, et al. Laparoscopic partial nephrectomy: evaluation of long-term oncological outcome. J Urol 2004 September; 172(3):871-3.
10. [Moinzadeh-2006] Moinzadeh A, Gill I S, Finelli A, Kaouk J, Desai M.

Laparoscopic partial nephrectomy: 3-year followup. J Urol 2006 February; 175(2):459-62.

11. [Hollenbeck-2006] Hollenbeck B K, Taub D A, Miller D C, Dunn R L, Wei J T. National utilization trends of partial nephrectomy for renal cell carcinoma: a case of underutilization Urology 2006 February; 67(2):254-9.

12. [Huang-2009] Huang W C, Elkin E B, Levey A S, Jang T L, Russo P. Partial nephrectomy versus radical nephrectomy in patients with small renal tumors—is there a difference in mortality and cardiovascular outcomes? J Urol 2009 JanUary; 181(1):55-61; discussion—2.
13. [Thompson-2008] Thompson R H, Boorjian S A, Lohse C M, Leibovich B C, Kwon E D, Cheville J C, et al. Radical nephrectomy for pT1a renal masses may be associated with decreased overall survival compared with partial nephrectomy. J Urol 2008 February; 179(2):468-71; discussion 72-3.
14. [Zini-2009] Zini L, Perrotte P, Capitanio U, Jeldres C, Shariat S F, Antebi E, et al. Radical versus partial nephrectomy: effect on overall and noncancer mortality. Cancer 2009 Apr. 1; 115(7):1465-71.
15. Stolka P J, Keil M, Sakas G, McVeigh E R, Taylor R H, Boctor E M, “A 3D-elastography-guided system for laparoscopic partial nephrectomies”. SPIE Medical Imaging 2010 (San Diego, Calif./USA)
61. [Jemal-2008] Jemal A, Siegel R, Ward E, et al. Cancer statistics, 2008. CA Cancer J Clin 2008; 58:71-96. SFX
62. [Hock-2002] Hock L, Lynch J, Balaji K. Increasing incidence of all stages of kidney cancer in the last 2 decades in the United States: an analysis of surveillance, epidemiology and end results program data. J Urol 2002; 167:57-60. Ovid Full Text Bibliographic Links
63. [Volpe-2005] Volpe A, Jewett M. The natural history of small renal masses. Nat Clin Pract Urol 2005; 2:384-390. SFX
[Ismail-2011] Ismail M M, Taguchi K, Xu J, Tsui B M, Boctor E, “3D-guided CT reconstruction using time-of-flight camera,” Accepted in SPIE Medical Imaging 2011
[Xu-2010] Xu, J.; Taguchi, K.; Tsui, B. M. W.; “Statistical Projection Completion in X-ray CT Using Consistency Conditions,” Medical Imaging, IEEE Transactions on, vol. 29, no. 8, pp. 1528-1540, August 2010

1. A system for providing visual information for use in guiding the use of an instrument relative to a body during a procedure, comprising:
a body imaging system configured to image a body along an image plane,
a camera configured to observe a region of imaging during operation of said imaging system, and
a projector aligned with said image plane,
wherein said camera is aligned off-axis relative to said image plane;
wherein said projector is configured to project an image that indicates a location of the image plane;
wherein an image recorded by said camera is displayed on a screen, and
wherein the system is configured to superimpose guidance information on the camera display such that intersection of the image plane and the plane formed by the superimposed guidance forms a line that corresponds to a desired trajectory of the instrument.

2. A system according to 1 wherein the display screen is configured to display the image produced by the imaging system together with the image recorded by said camera.

3. (canceled)

4. A system for providing visual information for use in guiding the use of an instrument relative to a body during a procedure, comprising:
a body imaging system configured to image a body along an image plane,
a camera configured to observe a region of imaging during operation of said imaging system, and aligned with said image plane, and
a projector aligned off-axis relative to said image plane;
wherein an image recorded by said camera is displayed on a screen, and
wherein information indicating a location of the image plane is superimposed on the displayed camera image;
wherein the projection system projects a line corresponding to a desired instrument trajectory.

5. (canceled)

6. A system for providing visual information for use in guiding the use of an instrument relative to a body during a procedure, comprising:
a body imaging system configured to image a body along an image plane,
a camera configured to observe a region of imaging during operation of said imaging system, and
a projector configured to project an image,
wherein one of said camera and said projector is aligned with said image plane,
wherein another of said camera and said projector is aligned off-axis relative to said image plane;
wherein said projector is configured to project an image corresponding to a calculated location of a shadow cast by said instrument when said instrument is located in a proper location and orientation for said procedure.

7. (canceled)

8. (canceled)

9. (canceled)

10. (canceled)

11. (canceled)

12. A system for providing visual information for use in guiding the use of an instrument relative to a body during a procedure, comprising:
a body imaging system configured to image a body along an image plane,
a first camera configured to observe a region of imaging during operation of said imaging system, and
a second camera configured to observe a region of said imaging during operation of said imaging system, said first and second cameras configured and located for stereoscopic observation of said region of imaging,
said first and second cameras configured to actively track the location and movement of said instrument, and
said system configured to display guidance information concerning the location and orientation of the instrument relative to a desired location and orientation of the instrument for said procedure.

13. (canceled)

14. A system according to claim 12, wherein said guidance information is projected onto the patient.

15. (canceled)

16. A system according to claim 12, wherein said guidance information is projected as an overlay on a projection of the image captured by the imaging system.

17. (canceled)

18. A system according to claim 12, wherein the guidance information is registered to the projected image or to the subject.

19. A system according to claim 12, wherein the guidance information is location independent.

20. (canceled)

21. A system according to claim 12, further configured to project guidance concerning placement of the imaging system.

22. A system according to claim 12, wherein images acquired by the imaging system are registered with images recorded with the camera, and the projector uses said registered image to project guidance information for improved visualization of a selected target.

23. A system according to claim 1, configured to reference stored prior images of said body to provide guidance concerning for placement of the imaging system for optimal imaging of a desired target.

24. A system according to claim 1, configured to display said guidance information is superimposed over live images from said imaging system.

25. (canceled)

26. A system according to claim 1, configured to superimpose alignment lines corresponding to desired instrument location and orientation over single, stereo or multiple camera views, and configured to project instrument alignment lines denoting current instrument location and orientation.

27. (canceled)

28. (canceled)

29. A system according to claim 1, wherein a thickness of said guidelines may be varied based on detected instrument dimensions, distance to projector, distance to the body.

30. A system according to claim 12, further comprising:
a projector assembly configured to project, from two different angles, an image from said imaging system,
wherein said system is configured to display guidance information concerning the location and orientation of the instrument relative to a desired location and orientation of the instrument for said procedure using intersecting shadows.

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. A system according to claim 1, wherein the projector is configured to project a visual interface on a surface, and wherein the camera system is configured to track user interaction with the visual interface according to which the user may provide instructions to the system by interacting with the visual interface.

57. A system according to claim 1, wherein the projector accounts for the three-dimensional structure of a projection surface when projecting a visual interface on said surface.

58. (canceled)

59. (canceled)

60. (canceled)

61. (canceled)

62. A system according to claim 1, further comprising a display system that maintains registration with the imaging system and which is configured for both visualization and guidance.

63. A system according to claim 1, wherein said imaging system has a detachable display configured to show pre-operative information based on its position in space.

64. (canceled)

65. (canceled)

66. (canceled)

67. A system according to claim 1, further comprising a sterile sheath for at least one of said camera and projector, said sheath containing a transparent solid plastic window to permit projection and camera recording.

68. A method according to claim 3, comprising projecting said image onto a drape covering at least a portion of said subject.

69. A method according to claim 68, wherein said drape is selected from the group consisting of: transparent to structured light, IR transparent, and wavelength-specific.

70. A method according to claim 68, wherein said drape comprises a detectable reference frame sufficient to allow direct surface tracking and registration.

71. (canceled)

72. (canceled)

73. A method according to claim 3, further comprising time multiplexing the projection image with the camera to optimize projection for tracking, guidance, and surface.

74. A method according to claim 3, further comprising time multiplexing or spatially modulating the projection pattern.

75. A method according to claim 3, further comprising projecting an adaptive pattern according to space and/or time, wherein said adaptive pattern is selected from the group consisting of: changing spatial frequencies of the pattern according to surface distance, structure size and/or camera resolution, changing pattern color to adapt to surface properties, and randomizing patterns over time.

76. (canceled)

77. (canceled)

78. (canceled)

79. (canceled)
